(19) United States
(12) Patent Application Publication
(10) Pub. No.: US 2011/0182979 A1
(43) Pub. Date: Jul. 28, 2011

(54) In-Vivo Non-Viral Gene Delivery of Human Vascular Endothelial Growth Factor Following Islet Transplantation

(75) Inventors: Masayuki Shimoda, Irving, TX (US); Shuyuan Chen, Allen, TX (US); Hirofumi Noguchi, Dallas, TX (US); Shinichi Matsumoto, Arlington, TX (US); Paul A. Graybum, Dallas, TX (US)

(73) Assignee: BAYLOR RESEARCH INSTITUTE, Dallas, TX (US)

(21) Appl. No.: 13/014,926

(22) Filed: Jan. 27, 2011

Related U.S. Application Data
(60) Provisional application No. 61/298,824, filed on Jan. 27, 2010.

(51) Int. Cl. A61K 9/127; A61K 48/00; A61K 38/18; C12N 5/00; A61K 35/39; A61K 31/713; A61P 3/10; A61P 1/16

(52) U.S. Cl. 424/450; 514/44 R; 514/8.1; 435/375; 424/93.7; 514/44 A

(57) ABSTRACT

An ultrasound-mediated gene transfer method named Ultrasound Targeted Microbubble Destruction (UTMD) for the delivery of human vascular endothelial growth factor (hVEGF) gene to transplanted islets and the surrounding tissue is described herein. The delivery of hVEGF promotes islet revascularization and survival. The inventors, first transplanted human islets were transplanted into diabetic nude mice liver followed by the induction of non-viral plasmid vectors encoding hVEGF or Green Fluorescent Protein (GFP) gene in the host liver by UTMD. Transplantation without gene delivery was also performed as a control. Blood glucose, serum human insulin, C-peptide levels and the revascularization in graft islets were evaluated. The findings of the method of the present invention indicated that hVEGF gene delivery to host liver using UTMD promoted islet revascularization after islet transplantation and improved the restoration of euglycemia.

### CROSS-REFERENCE TO RELATED APPLICATIONS

This application claims priority to U.S. Provisional Application Ser. No. 61/298,824, filed Jan. 27, 2010, the entire contents of which are incorporated herein by reference.

### STATEMENT OF FEDERALLY FUNDED RESEARCH

This invention was made with U.S. Government support under Contract Nos. R01 HL072430-01 and 2P01 DK58398 awarded by the National Institutes of Health (NIH). The government has certain rights in this invention.

### TECHNICAL FIELD OF THE INVENTION

The present invention relates in general to the field of gene delivery, and more particularly, to the development of an ultrasound-mediated gene transfer method for the delivery of human vascular endothelial growth factor (hVEGF) gene to transplanted islets and the surrounding tissue to promote islet revascularization and survival.

### REFERENCE TO A SEQUENCE LISTING

The present application includes a Sequence Listing filed separately as required by 37 CFR 1.821-1.825.

### BACKGROUND OF THE INVENTION

Without limiting the scope of the invention, its background is described in connection with gene delivery methods to improve efficacy of transplanted cells and tissues and in the treatment of diseases.

U.S. Patent Application No. 20080114287 (Lai and Lan, 2008) describes a method for delivery of agents such as genes, plasmids, and other active DNA-related molecules useful for treatment peritoneal disease, including peritoneal fibrosis or postoperative adhesion specifically using an ultrasound-triggered disruption of inducible Smad7 gene-bearing microbubble system. The invention, provides a source of microbubbles containing one or more inducible Smad7 genes, DNA molecules, or plasmids for treatment of peritoneal disease, followed by perfusion of the peritoneal region of the patient with the microbubbles; providing ultrasonic energy to the abdominal region sufficient to cause transfection of the one or more inducible Smad7 genes, DNA molecules or plasmids from the microbubbles into the peritoneal region to penetrate peritoneal tissue found therein.

U.S. Pat. No. 7,374,390 issued to Oh et al. (2008), discloses compositions and methods of use to normalize blood glucose levels of patients with type 2 diabetes. The invention includes a plasmid comprising a chicken 0 actin promoter and enhancer; a modified GLP-1 (7-37) cDNA (pβGLP1), carrying a furin cleavage site, which is constructed and delivered into a cell for the expression of active GLP-1.

U.S. Patent Application No. 20090209630, filed by Coleman, et al. (2009), discloses a novel approach for efficient delivery of angiogenic factors to the cardiac and peripheral vasculature that avoids problems with toxicity inherent to existing delivery technologies. Vectors carrying coding sequences for angiogenic agents including Del-1 or VEGF, or both, can be formulated with poloxamers or other polymers for delivery into ischemic tissue and delivered to areas of peripheral ischemia in a flow to no-flow pattern and to the heart by retrograde venous perfusion.

### SUMMARY OF THE INVENTION

The present invention uses Ultrasound Targeted Microbubble Destruction (UTMD) for gene delivery of human vascular endothelial growth factor (hVEGF) gene (SEQ. ID NO: 9) to transplanted islets and the surrounding tissue for promotion of islet revascularization and survival. A number of human islets were transplanted into diabetic nude mice liver followed by induction of non-viral plasmid vectors encoding hVEGF (SEQ. ID NO: 9) or Green Fluorescent Protein (GFP) gene (SEQ. ID NO: 11) in the host liver by UTMD. Transplantation without gene delivery was performed as a control. Using the present invention it was possible to stabilize blood glucose, serum human insulin, C-peptide levels and the revascularization in graft islets.

In one embodiment, the present invention includes a composition for ultrasound-targeted microbubble destruction (UTMD) in one or more liver cells, a liver or an islet cell transplanted into the liver comprising: one or more pre-assembled liposome plasmid DNA (pDNA) microbubble complexes, wherein the microbubble comprises a lipid shell enclosing a gas and a pDNA comprising a constitutive promoter sequence or an inducible promoter sequence operably linked to a human vascular endothelial growth factor (hVEGF), wherein an ultrasound disruption of the one or more microbubbles in the one or more liver cells, the liver or the cells transplanted into the liver delivers the pDNA into the one or more liver cells, the liver or the cells transplanted into liver at a location of the ultrasound disruption express hVEGF, wherein the composition improves the efficacy of the one or more transplanted islet cells. In one aspect, the lipid shell comprises one or more additional bioactive agents selected from the group consisting of naked DNA, siRNA, plasmids, proteins, viral vectors and drugs. In another aspect, the gas is a perfluorocarbon gas. In another aspect, the inducible promoter comprises a tissue-specific regulatory element. In another aspect, the efficacy of the islet transplantation is measured by improved revascularization, improved islet cell function, increased vessel density or combinations thereof. In another aspect, the hVEGF is a recombinant hVEGF. In another aspect, one or more agents may be co-administered with the composition, wherein the agents are selected from the group consisting of an anti-apoptotic agent, an anti-inflammatory agent, a JNK inhibitor, a GLP-1, a tacrolimus, a sirolimus, an anakinra, a Dervin polyamide or combinations thereof.

Another embodiment of the present invention is a composition for regenerating transplanted islet cells in a liver or a transplanted liver using ultrasound-targeted microbubble destruction (UTMD) comprising microbubbles comprising a naked plasmid DNA encoding a human vascular endothelial growth factor (hVEGF), wherein the microbubbles comprise lipids that release the hVEGF by ultrasound disruption in the liver or the transplanted liver. In one aspect, the hVEGF is a recombinant hVEGF. In another aspect, the constitutive promoter sequence or an inducible promoter sequence operably linked to a human vascular endothelial growth factor, e.g., an insulin or a cytomegalovirus (CMV) promoter.

Another embodiment of the present invention is a method for promoting revascularization, improving function, increasing vessel density and efficacy of one or more transplanted cells or grafted cells in vivo and in situ in subject comprising the step of: delivering an effective amount of a microbubble composition comprising a naked plasmid DNA encoding a human vascular endothelial growth factor (hVEGF), wherein the microbubbles comprise lipids that release the hVEGF by an ultrasound disruption in the one or more transplanted or grafted cells, wherein the released hVEGF promotes revascularization, improves function, vessel density and efficacy of the one or more transplanted or grafted cells. In one aspect, the one or more transplanted or grafted cells comprise islet cells. In another aspect, the subject is a healthy subject, a diabetic subject or a subject in need of one or more transplanted or grafted cells.

Yet another embodiment of the present invention is a method of improving vascularization, increasing vessel density and efficacy of one or more transplanted islet cells in the liver of a patient comprising the steps of: injecting the patient with a naked plasmid DNA microbubble complex comprising a plasmid expressing a human vascular endothelial growth factor (hVEGF) gene under the control of cytomegalovirus (CMV) promoter, wherein the injection is done in the liver of the patient; delivering the pDNA to the one or more transplanted islet cells in the liver; and maintaining the one or more transplanted islet cells under conditions effective to express the hVEGF gene (SEQ. ID NO: 9), wherein the expression of the hVEGF causes improved vascularization, increased vessel density and efficacy of the one or more transplanted islet cells. In one aspect, the method further comprises optional co-administration of one or more agents, wherein the agents are selected from the group consisting of an anti-apoptotic agent, an anti-inflammatory agent, a JNK inhibitor, a GLP-1, a tacrolimus, a sirolimus, an anakinra, a Dervin polyamide or combinations thereof. In another aspect, the microbubble comprises a pre-assembled liposome-naked plasmid DNA (PDNA) complex. In another aspect, the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

Another embodiment of the present invention is a method of treating diabetes or promoting euglycemia in a patient comprising the steps of: identifying the patient in need of treatment against the diabetes or promotion of the euglycemia; transplanting one or more islet cells by infusing the patient's liver with one or more islet cells, wherein the one or more transplanted islet cells produce insulin for the treatment of the diabetes or for the promotion of the euglycemia; injecting an effective amount of a microbubble composition comprising a naked plasmid DNA (pDNA) encoding a human vascular endothelial growth factor (hVEGF), wherein the microbubbles comprise lipids that release the hVEGF by an ultrasound disruption in the one or more transplanted islet cells, wherein the released hVEGF promotes revascularization, improves function, vessel density and efficacy of the one or more transplanted islet cells; and treating the diabetes or promoting the euglycemia by the production of insulin by the one or more transplanted islet cells. In one aspect, the hVEGF is a recombinant hVEGF.

Yet another embodiment of the present invention includes a composition for ultrasound-targeted microbubble destruction (UTMD) in a body organ comprising: a pre-assembled liposome-bioactive agent complex in contact with a microbubble, wherein the bioactive agents are selected from the group consisting of a naked plasmid DNA (pDNA), a siRNA, one or more plasmids, proteins, viral vectors and drugs, wherein the pre-assembled liposome-bioactive agent complex may express a gene under the control of one or more promoters, wherein disruption of the microbubble with ultrasound in the body organ at a target site delivers the bioactive agent at a location of the ultrasound disruption. In another aspect, the one or more cells comprise transplanted islet cells. In another aspect, the body organs comprise liver, pancreas, kidney, lungs, or heart. In another aspect, the body organ is the liver. In another aspect, the bioactive agent is a pDNA. In another aspect, the pre-assembled liposome-bioactive agent complex expresses a recombinant human vascular endothelial growth factor (hVEGF) gene under the control of a cytomegalovirus (CMV) promoter. In another aspect, the pre-assembled liposome-nucleic acid complex comprises cationic lipids, anionic lipids or mixtures and combinations thereof. In another aspect, the microbubbles are disposed in a pharmaceutically acceptable vehicle. In another aspect, the pre-assembled liposome-bioactive agent complex comprise: 1,2-dipalmitoyl-sn-glycero-3-phosphatidylcholine and 1,2-dipalmitoyl-sn-glycero-3-phosphatidylethanolamine glycerol mixed with a plasmid. In another aspect, the composition promotes revascularization, improves function, increases vessel density and efficacy of the one or more transplanted islet cells. In another aspect, the composition further comprises an optional coating.

### BRIEF DESCRIPTION OF THE DRAWINGS

For a more complete understanding of the features and advantages of the present invention, reference is now made to the detailed description of the invention along with the accompanying figures and in which:

FIGS. 1A-1D shows UTMD to mouse liver via ileocecal vein: (1A) infusion into ileocecal vein. Arrow: ileocecal vein, Arrowhead: hemoclip, Asterisk: 27 G wing needle, (1B) setup of UTMD. An ultrasound probe was put on the upper abdomen. Arrow: probe, (1C-1D) images of ultrasound, (1C) mouse liver without microbubbles. The liver was displayed at low echo level, (1D) mouse liver after injecting microbubbles. White opacification was detected before microbubble destruction (left), and it disappeared after destruction (right);

FIGS. 2A-2H shows hVEGF gene (SEQ. ID NO: 9) delivery by UTMD. Mouse liver was removed after UTMD with hVEGF or GFP plasmid and examined by immunohistochemistry and RT-PCR. (2A-2E) immunohistochemical analysis of mouse liver. (2A) 3 days, (2B) 10 days, and (2C) 14 days after UTMD with hVEGF. In FIGS. 2A and 2B, hVEGF expression was detected, especially near a portal vein. In FIG. 2C hVEGF expression very low, (2D) normal mouse liver as a control wherein hVEGF was not expressed, (2E) GFP induced mouse. GFP expression was detected. P: portal vein, Green: (2A-2D) VEGF or (2E) GFP, Blue: DAPI. Magnification: ×100. Scale bar: 100 μm, (2F) RT-PCR of hVEGF-UTMD mouse liver. hVEGF was strongly expressed in UTMD treated group (No. 1-3) whereas it was not detected in nontransfected control (No. 4). All mice expressed endogenous (mouse) VEGF. GAPDH was used as a standard. mVEGF: mouse VEGF, (2G) serum hVEGF level of UTMD treated mice. hVEGF was detected up to 14 days after UTMD. ND: not determined, (2H) organ specificity of hVEGF expression. Various organs were harvested 3 days after UTMD with hVEGF plasmid and hVEGF expression was examined by PT-PCR. hVEGF expression was strongly detected in liver, whereas other organs hardly expressed. The slight expression was seen in right kidney, because this organ was exposed to ultrasound anatomically;

FIGS. 3A-3D effect of UTMD and hVEGF on liver: (3A) serum AST (solid line) and ALT (broken line) levels after UTMD. Arrow: Time of UTMD treatment, (3B) histological analysis of liver after UTMD (HE staining) Original magnification: ×100, (3C) vessel density of liver at day 32 after treatment. There was no significant difference among 3 groups, (3D) the ratio of the weight of left lobe of liver to the body weight at day 32 after UTMD. There was no significant difference among 3 groups;

FIGS. 3E-3H effect of hVEGF on endogenous pancreas: hVEGF gene (SEQ. ID NO: 9) was delivered to liver of STZ induced diabetic mice by UTMD (STZ+VEGF group). They were compared with Non-treatment mice (Normal group) and STZ-induced diabetic mice (STZ group): (3E) non fasting blood glucose levels after treatment in STZ (Broken line) and STZ+VEGF (Solid line) groups. Arrow: Time of UTMD treatment, (3F) representative sections of pancreas stained with Insulin, CD31 and DAPI at ×100. Scale bar: 100 μm, (3G) beta cell mass in pancreas at day 20 after treatment, (3H) vessel density in islets at day 20 after treatment. VEGF did not affect on the beta cell mass and vessel density. Asterisk: p<0.01. NS: not significant;

FIGS. 4A-4C shows the hVEGF expression in the graft islet and surrounding tissue after islet transplantation and UTMD Immunohistochemistry of mouse liver 3 days after human islet transplantation followed by UTMD with hVEGF. hVEGF expression was mostly detected in the surface and outer part of islets as well as the surrounding tissue: (4A) Green: hVEGF, Red: human insulin, (4B) human Glucagon, (4C) Vimentin, Blue: DAPI. Magnification: φ200, White line: Border of graft islet, P: Lumen of portal vein. Scale bar: 100 μm;

FIGS. 5A-5D shows the blood glucose level in 3 groups: 500 human islets were transplanted into liver of STZ induced diabetic nude mice. Then UTMD was performed with GFP (5B: GFP group, n=7) or hVEGF (5C: VEGF group, n=11) plasmid. UTMD was not conducted in no UTMD group (5A: n=8), (5D) average of blood glucose level in 3 groups. Broken line: no UTMD group, Dotted line: GFP group, Solid line: VEGF group. preTX: pretreatment;

FIGS. 5E and 5F show the Kaplan-Meyer plot of graft survival of 3 groups. The day 7 was set as a baseline and the p value for the survival curve was determined by the log-rank test. (5E) There were significant differences in no UTMD group vs. VEGF group (p<0.05) and in GFP group vs. VEGF group (p<0.05), (5F) IPGTT at day 31 after treatment in 3 groups. Broken line: no UTMD group, Dotted line: GFP group, Solid line: VEGF group;

FIGS. 6A and 6B show the (6A) serum human insulin and (6B) C-peptide levels in 3 groups. Blood was collected from each mouse at day 32 after treatment. Asterisk: p<0.025; and

FIGS. 7A-7F shows the revascularization of graft islets and beta cell mass at day 32 after treatment. FIGS. 7A-7D Representative sections stained with human insulin, CD31 and DAPI at φ200. (7A) no UTMD (7B) GFP (7C) VEGF group, respectively. Several vessels were detected in the transplanted islet in VEGF group, (7D) normal human islets in pancreas as a control. Red: human Insulin, Green: CD31, Blue: DAPI, Arrow: Vessels in islets, (7E) vessel density in transplanted islets. Mean values in 3 groups for vessel density at day 10 and 32 after treatment were shown. Original human islets were used as a control. At day 32, the vessel density of VEGF group was significantly higher than both no UTMD and GFP group; however it was significantly lower than the original islets, (7F) beta cell mass in the left lobe of liver at day 32 after treatment. Asterisk: p<0.01. Double asterisk: p<0.005.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an,” and “the” are not intended to refer to only a singular entity, but include the general class of which a specific example may be used for illustration. The terminology herein is used to describe specific embodiments of the invention, but their usage does not delimit the invention, except as outlined in the claims.

The term “diabetes” as described in embodiments of the present invention refers to the chronic disease characterized by relative or absolute deficiency of insulin that results in glucose intolerance. The term “diabetes” is also intended to include those individuals with hyperglycemia, including chronic hyperglycemia, hyperinsulinemia, impaired glucose homeostasis or tolerance, and insulin resistance.

The term “insulin” as used herein shall be interpreted to encompass insulin analogs, natural extracted human insulin, recombinantly produced human insulin, insulin extracted from bovine and/or porcine sources, recombinantly produced porcine and bovine insulin and mixtures of any of these insulin products. The term is intended to encompass the polypeptide normally used in the treatment of diabetics in a substantially purified form but encompasses the use of the term in its commercially available pharmaceutical form, which includes additional excipients. The insulin is preferably recombinantly produced and may be dehydrated (completely dried) or in solution.

The term “islet cell (s)” as used throughout the specification is a general term to describe the clumps of cells within the pancreas known as islets, e.g., islets of Langerhans. Islets of Langerhans contain several cell types that include, e.g., β-cells (which make insulin), α-cells (which produce glucagons), γ-cells (which make somatostatin), F cells (which produce pancreatic polypeptide), enterochromaffin cells (which produce serotonin), PP cells and D1 cells. The term “stem cell” is an art recognized term that refers to cells having the ability to divide for indefinite periods in culture and to give rise to specialized cells. Included within this term are, for example, totipotent, pluripotent, multipotent, and unipotent stem cells, e.g., neuronal, liver, muscle, and hematopoietic stem cells.

The term “gene” is used to refer to a functional protein, polypeptide or peptide-encoding unit. As will be understood by those in the art, this functional term includes both genomic sequences, cDNA sequences, or fragments or combinations thereof, as well as gene products, including those that may have been altered by the hand of man. Purified genes, nucleic acids, protein and the like are used to refer to these entities when identified and separated from at least one contaminating nucleic acid or protein with which it is ordinarily associated

As used herein, the term “vector” is used in reference to nucleic acid molecules that transfer DNA segment(s) from one cell to another. The vector may be further defined as one designed to propagate specific sequences, or as an expression vector that includes a promoter operatively linked to the specific sequence, or one designed to cause such a promoter to be introduced. The vector may exist in a state independent of the host cell chromosome, or may be integrated into the host cell chromosome

As used herein, the term “promoter” is defined as a DNA sequence recognized by the synthetic machinery of the cell, or introduced synthetic machinery, required to initiate the specific transcription of a gene. As used herein, the term “under transcriptional control” or “operatively linked” is defined as the promoter is in the correct location and orientation in relation to the nucleic acid to control RNA polymerase initiation and expression of the hVEGF gene (SEQ. ID NO: 9).

As used herein, the term “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g., α-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes so-called “peptide nucleic acids,” which comprise naturally-occurring or modified nucleic acid bases attached to a polyamide backbone. Nucleic acids can be either single stranded or double stranded.

The term “transfection” as used herein refers to the introduction of foreign DNA into eukaryotic cells. Transfection may be accomplished by a variety of means known to the art including, e.g., calcium phosphate-DNA co-precipitation, DEAE-dextran-mediated transfection, polybrene-mediated transfection, electroporation, microinjection, liposome fusion, lipofection, protoplast fusion, retroviral infection, and biolistics. Thus, the term “stable transfection” or “stably transfected” refers to the introduction and integration of foreign DNA into the genome of the transfected cell. The term “stable transfectant” refers to a cell which has stably integrated foreign DNA into the genomic DNA. The term also encompasses cells which transiently express the inserted DNA or RNA for limited periods of time. Thus, the term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes.

The term “transient transfectant” refers to cells which have taken up foreign DNA but have failed to integrate this DNA.

As used herein, the term “in vivo” refers to being inside the body. The term “in vitro” used as used in the present application is to be understood as indicating an operation carried out in a non-living system.

The term “liposome” as used herein refers to a capsule wherein the wall or membrane thereof is formed of lipids, especially phospholipid, with the optional addition therewith of a sterol, especially cholesterol.

As used herein, the term “treatment” or “treating” means any administration of a compound of the present invention and includes (1) inhibiting the disease in an animal that is experiencing or displaying the pathology or symptomatology of the diseased (i.e., arresting further development of the pathology and/or symptomatology), or (2) ameliorating the disease in an animal that is experiencing or displaying the pathology or symptomatology of the diseased (i.e., reversing the pathology and/or symptomatology).

The present invention describes an ultrasound targeted microbubble destruction (UTMD) method for the non-viral hVEGF gene (SEQ. ID NO: 9) delivery to transplanted islet cells for the promotion of islet revascularization and survival. The present inventors induced non-viral plasmid vectors encoding hVEGF (SEQ. ID NO: 10) or Green Fluorescent Protein (GFP) (SEQ. ID NO: 12) in host diabetic nude mice livers, with previously transplanted islet cells. The results of studies conducted by the inventors showed a restoration of euglycemia and improved islet revascularization.

Islet transplantation is a promising treatment for type 1 diabetes, however, the efficacy of transplantation needs to be improved because currently multiple transplantation is required to achieve insulin free status [1, 2]. It was shown that gene delivery to islets can improve the function and survival of islets [3-6], but all previous studies used viral vectors. Viral vector has high efficacy to deliver genes, but adverse events have been related to enhancer-mediated mutagenesis of genomic DNA [7] or immunological responses to viral proteins [8].

In contrast, delivery of naked plasmid DNA (pDNA) does not transport toxic or immunogenic viral protein nor polymer particles in vivo. However, plasmid vectors have been limited by low transfection efficiency. To obtain high gene expression with pDNAs, the present inventors in the present invention describe an ultrasound-mediated gene transfer method known as ultrasound targeted microbubble destruction (UTMD). Microbubbles which consist of lipid shell encapsulating perfluorocarbon gas are injected into circulation. The pDNAs are incorporated in the lipid shell. Under ultrasound exposure, the microbubbles burst and create transient pores in membranes of surrounding cells, and pDNAs are inserted into the cells. UTMD has many desired characteristics of gene therapy including low toxicity, low immunogenicity, a potential for repeated application, organ specificity, and broad applicability to acoustically accessible organs. The inventors have previously demonstrated that delivery of human vascular endothelial growth factor (hVEGF) to rat myocardium by UTMD resulted in significant increases in myocardial capillary and arteriolar density [9]. Moreover, the inventors have previously reported that the UTMD technique enables the effective delivery of pDNAs to rat pancreatic beta cells in vivo [10, 11]. Lack of intra-islet microvasculature is one of the most important factors for loss of graft islets. Therefore, the inventors hypothesize that early loss of islet grafts could be attenuated by delivery of hVEGF gene (SEQ. ID NO: 9) to promote revascularization.

In the present invention, the inventors use a transplant model in which human islets were transplanted into mouse liver via portal vein. This model is similar to the clinical setting. hVEGF gene (SEQ. ID NO: 9) was delivered to the host liver by UTMD to examine whether it could facilitate revascularization and improve the survival and function of the transplanted islets.

The results of the studies conducted in the present invention showed a restoration of euglycemia in 13% of no UTMD and 14% in the GFP group, whereas 73% mice in VEGF group became euglycemic at day 30 (p<0.05 in no UTMD vs. VEGF). Serum human insulin and C-peptide were significantly higher in the hVEGF group at day 32 (Insulin: no UTMD −17±8; GFP—37±17; VEGF—109±26 pmol/L, respectively, p<0.05; C-peptide: no UTMD−68±38; GFP—115±58; VEGF—791±230 pmol/L, respectively, p<0.05). Vessel density in graft islets was significantly higher in the VEGF group (no UTMD; 169±36, GFP; 227±39, VEGF; 649±51 count/mm², respectively, p<0.05). The findings indicated that hVEGF gene (SEQ. ID NO: 9) delivery to host liver using UTMD promoted islet revascularization after islet transplantation and improved the restoration of euglycemia.

Human islets: Seven donor pancreata were procured from deceased multiorgan donors after obtaining consent for research through local Organ Procurement Organizations (Southwest Transplant Alliance, Dallas, Tex., LifeGift, Fort Worth, Tex.). Baylor Regional Transplant Institute Surgeons using the standardized surgical technique performed pancreas procurement [12]. Pancreata were preserved using the ductal injection method with ET-KYOTO solution (Otsuka Pharmaceutical Factory Inc., Naruto, Japan) followed by two-layer method until the islet isolation procedure [12]. Islet isolation was performed using the modified Ricordi method [13]. The Donor and isolation variables were as follows: Donor age 35.1±5.6 (y), Gender M/F=2/5, BMI 28.0±1.5, Cold ischemic time 171±21 (min), Purity 77±5(%), Viability 97±1(%), Stimulation Index 10.4±3.4. The isolated islets were preserved in CMRL-supplemented culture medium (Cellgro, Manassas, USA) containing 10% fetal bovine serum (FBS; Atlanta Biologicals, Lawrenceville, USA) at 4° C. overnight. A total of 500 islets of similar size (approximately 200 μm) were hand-picked for transplantation. Initial findings by the inventors showed that 10-20% of mice became euglycemic after transplantation of 500 islets. Therefore, the number was determined as a marginal.

Plasmid constructs: Plasmids expressing the hVEGF₁₆₅ gene under an enhanced CMV promoter (pCI-hVEGF) were made as follows: cDNA of hVEGF₁₆₅ was obtained as previously described [9], relevant portions and sequences incorporated herein by reference. Briefly, A full-length cDNA of the hVEGF₁₆₅ obtained from a healthy volunteer's blood was PCR amplified by using the following PCR primers that contain a restriction site (the restriction sites are underlined): primer 1 (XhoI) 5′-TTCCTCGAGAATGAACTTTCTGCTGCTGTCTTG-3′ (SEQ. ID NO: 1); primer 2 (SmaI) 5′-AAACCCGGGTCACCGCCTCGGCTTGTCA-3′ (SEQ. ID NO: 2). The DNA was digested with XhoI and SmaI and then ligated into the corresponding sites of pCI Mammalian Expression Vector (Promega). The plasmids were sequenced to confirm that no artifactual mutations were present. Plasmids encoding green fluorescent protein (GFP) gene under the enhanced CMV promoter (pCS2-GFP) were used as a control.

Manufacture of plasmid-containing lipid-stabilized microbubbles: Lipid-stabilized microbubbles were prepared as previously described by the inventors [10]. Briefly, a total of 200 μl of DPPC (1,2-dipalmitoyl-sn-glycero-3-phosphatidylcholine, Sigma, St Louis, USA, 2.5 mg/ml) and DPPE (1,2-dipalmitoyl-snglycero-3-phosphatidylethanolamine, Sigma, 0.5 mg/ml) solution, 5 μl of 10% human albumin and 50 μl of pure glycerol were added to 1.5 ml vials containing 2 mg of plasmids dissolved in 50 μl of Lipofectamine²⁰⁰⁰ (Invitrogen) and mixed well and incubated on room temperature for 10 min, the remaining headspace was filled with the perfluoropropane gas (Air Products, Inc., Allentown, USA) and then mechanically shaken for 30 s by a dental amalgamator (Vialmix™, Bristol-Myers Squibb Medical Imaging, N. Billerica, Mass., USA). The mean diameter and concentration of the microbubbles were measured with a particle counter (Beckman Coulter Multisizer III, Fullerton, Calif., USA). 10 μl of microbubble solution, which consisted of approximately 50 μg of the plasmids, was diluted with 0.3 ml phosphate-buffered solution (PBS) just before the injection and injected to each mouse.

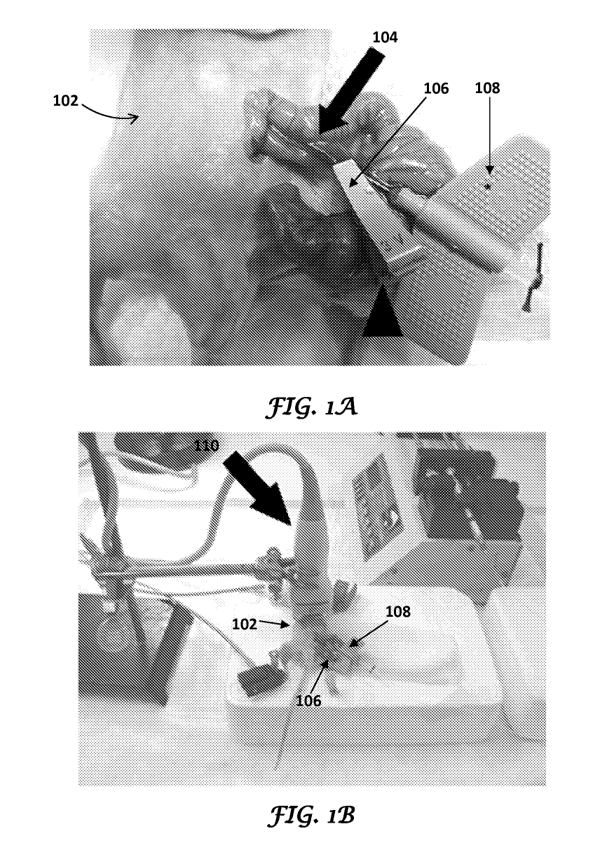

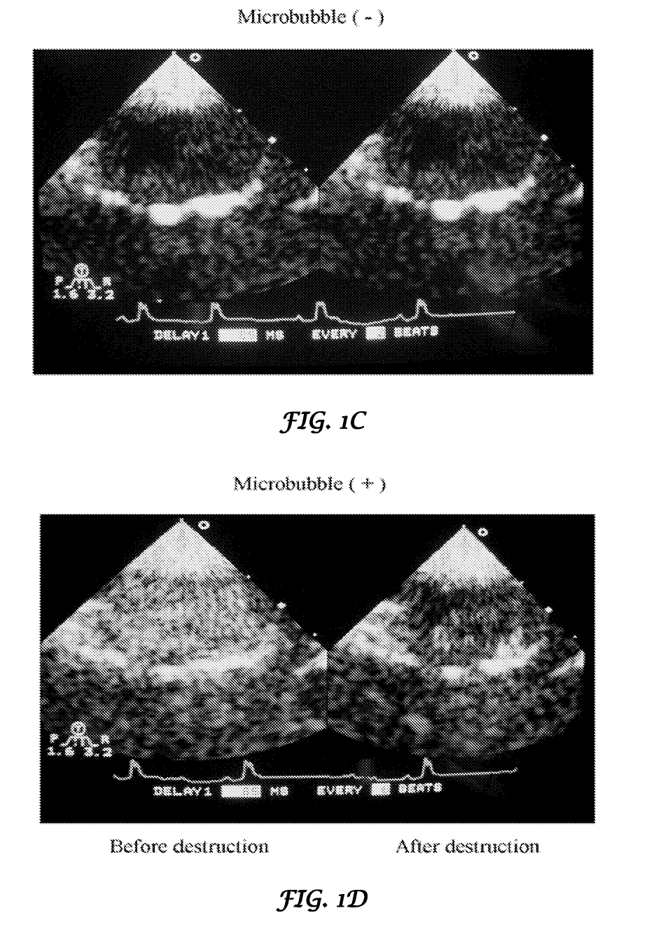

Animal protocol and UTMD: Animal studies were performed in accordance with National Institute of Health (NIH) recommendations and the approval of the institutional animal care and use committee. Streptozotocin (STZ, 150 mg/kg) was administered to male nude mice (8-13 weeks old, Harlan, Houston, USA) by intraperitoneal injection. The mice were considered to be diabetic after two consecutive measurements of blood glucose ≧350 mg/ml. Pre-transplantation diabetic status was not different among 3 groups (duration of diabetes: 4±1 day, pre-transplant blood glucose: 442±10 mg/dl). Mice 102 were anesthetized with intraperitoneal ketamine (100 mg/kg) and xylazine (10 mg/kg). The ileo-cecal vein 104 (a branch of portal vein) was pulled out by small midline section on lower abdomen so that the ultrasound probe 110 could be put on the upper abdomen (FIGS. 1A and 1B). The 27 G wing needle 108 was inserted and fixed with a hemoclip 106 (FIG. 1A). In this study, 26 mice received one of three treatments: 1. human islet transplantation alone without UTMD (no UTMD, n=8); 2. human islet transplantation and UTMD with plasmids encoding GFP gene (SEQ. ID NO: 11) (GFP, n=7); 3. human islet transplantation and UTMD with plasmids encoding hVEGF165 (VEGF, n=11).

Hand-picked human islets (n=500) were transplanted to the mouse liver through the ileo-cecal vein for each mouse. Then each mouse was randomly assigned to 3 groups. Microbubbles with plasmids were infused through the ileo-cecal vein in GFP and VEGF groups for 60-90 seconds via pump (Genie, Kent Scientific, Torrington, Conn., USA). During the infusion, ultrasound was directed to the liver using a commercially available ultrasound transducer (S3, Sonos 5500, Philips Ultrasound, Bothell, Wash., USA). Ultrasound was then applied in ultraharmonic mode (transmit 1.3 MHz/receive 3.6 MHz) at a mechanical index of 1.4. Four bursts of ultrasound were triggered to every fourth end-systole by electrocardiogram using a delay of 85 ms after the peak of the R wave. These settings have been shown to be optimal for plasmid delivery by UTMD using the particular instrument of the present invention [9-11]. Bubble destruction was visually apparent in all mice (FIGS. 1C and 1D). After infusion, the animals were allowed to recover. To study the efficacy of UTMD, UTMD was performed for normal nude mice as described above without islet transplantation. They were sacrificed at 1 to 28 post-treatment days for measuring serous human VEGF levels with ELISA kit (ALPCO Diagnostics, NH, USA). At day 3, the liver, pancreas, spleen, kidneys, stomach, lungs and heart were harvested for the assessment of gene expressions by RT-PCR.

To evaluate the extent and area of hVEGF (SEQ. ID NO: 10) expression in the transplanted islets, the inventors transplanted islets into normal nude mice via portal vein and then consecutively performed UTMD with hVEGF plasmids. Three days after the treatment, the liver was harvested and examined by immunohistochemistry.

Effect of UTMD on liver: To evaluate liver toxicity and unwanted effects of hVEGF (SEQ. ID NO: 10) such as excessive liver angiogenesis and uncontrolled liver growth, UTMD with hVEGF plasmid via portal vein was performed as described above. The blood was collected at different time points after UTMD. Samples were analyzed for the presence of alanine aminotransferase (ALT) and aspartate aminotransferase (AST) by enzyme assay kits (Thermo Scientific, MA, USA). Histological examination was performed on formalin-fixed mouse liver sections isolated at day 2, 7, 30 after UTMD by HE staining.

Assessment of the treated mice: Non-fasting glucose level of each mouse was measured with blood glucose test strip (Precision, Abbott, Ill., USA) up to 30 days post-transplantation. When two consecutive blood glucose level measurements were less than 200 mg/dl, restoration of euglycemia was considered to be present. Recurrence of diabetes was defined as return of the blood glucose to more than 200 mg/dl. At day 31, intraperitoneal glucose tolerance test (IPGTT) was performed. After overnight fasting, glucose (2 g/kg) was intraperitoneally injected. The blood glucose levels were measured for 120 minutes after injection. Animals were sacrificed at day 32. The harvested liver was weighed and examined by immunohistochemistry. The blood was collected for measuring human insulin and human C-peptide levels with ELISA kit (ALPCO Diagnostics).

RT-PCR: The tissue samples were frozen in liquid nitrogen and stored at −80° C. Total RNA was prepared from TRIzol (Invitrogen, Carlsbad, USA) according to the manufacturer's instructions and was reversetranscribed using SuperScript III First-Strand Synthesis System (Invtrogen). PCR was performed in 20 μl volume containing 1 μl of cDNA, 10 μl of HotStarTaq Master Mix (Qiagen), and 10 pmol of each primer:

GAPDH (for both human and mouse):
(SEQ. ID NO: 3)
5′-CCCTTCATTGACCTCAACTACATG-3′(sense);
(SEQ. ID NO: 4)
5′-TTCCATTGATGACAAGCTTCCC-3′(antisense);
hVEGF:
(SEQ. ID NO: 5)
5′-AAGGAGGAGGGCAGAATCAT-3′(sense);
(SEQ. ID NO: 6)
5′-ATCTGCATGGTGATGTTGGA-3′(antisense);
Mouse VEGF:
(SEQ. ID NO: 7)
5′-ACGACAGAAGGAGAGCAGAAGT-3′(sense);
(SEQ. ID NO: 8)
5′-CATGGTGATGTTGCTCTCTGAC-3′.

The PCR conditions included denaturation at 95° C. for 5 minutes, followed by 25-35 cycles of amplification by sequential denaturation at 94° C. for 30 seconds and primer annealing as well as strand extension for 1 minute. The RT-PCR products were then analyzed on 2% agarose gels.

Immunohistochemistry: The collected tissues were fixed in 4% paraformaldehyde at 4° C. overnight and equilibrated in 30% sucrose at 4° C. overnight for cryoprotection. The tissues were cryopreserved with Tissue Tek optimal cutting temperature (OCT) compound (Sakura Finetek USA, Torrance, Calif., USA) at −80° C. Sections were cut at 10 μm increments and placed onto positively charged microscope slides. Sections were permeabilized with 0.1% Triton X 100 for 3 minutes and incubated for 30 minutes in 20% Aquablock (East Coast Biologics, North Berwick, Mass., USA) for blocking Antibodies to the following antigens were used: guinea pig anti-human insulin (1:200, Abcam, Cambridge, Mass.), rabbit anti-glucagon (1:20, Millipore, Billerica, Mass., USA), goat anti-Vimentin (1:10, Sigma-Aldrich, St. Louis, Mo.), mouse anti-human CD31 (1:50, BD Biosciences, San Jose, Calif.), rat anti-mouse PECAM-1 (1:100, BD Biosciences), and mouse anti-human VEGF (1:100, Millipore). The sections with these first antibodies were incubated at 4° C. overnight. The antigens were visualized using appropriate secondary antibodies conjugated with fluorescin isothiocyanate (FITC), Cy3 (Jackson ImmunoResearch Laboratories, West Grove, Pa.), Alexa-fluor-488, and Alexa-fluor-568 (Invitrogen). Secondary antibodies were used at concentrations recommended by the manufacturer. Then, 4′,6-diamidino-2-phenylindole (DAPI) was added to the sections for nuclear staining. In this study, the second antibodies against human CD31 and mouse PECAM-1 were labeled with the same fluorescence. Islets were visualized on a fluorescent microscope and images were analyzed using MetaMorph software (Molecular Devices, Sunnyvale, Calif.). Insulin positive beta cell mass in the host liver (left lobe) at day 32 after treatment was measured as previously described [14].

Capillary density measurement: Intrainsular CD31 positive vessels visualized by immunohistochemistry were considered as capillaries. The capillaries were counted by the use of a fluorescent microscopy at a magnification of 200. At least two photomicrographs of the insulin positive graft islets in the host liver were taken from each mouse, giving a total of more than 30 islets per group. The area of each islet was measured with ImageJ software (National Institutes of Health, Bethesda, USA). Pre-isolation human islets were assessed as a control. Capillary density was expressed as the number per mm². The investigator reading the capillary density was blinded to treatment group. The vessel density of mouse liver was measured with a similar procedure.

Effect of VEGF on endogenous pancreas: To investigate the effect of circulating hVEGF on endogenous pancreas, UTMD with hVEGF plasmid to the liver was performed to STZ-induced diabetic mice. Normal mice and STZ-induced diabetic mice were used as controls. Non-fasting blood glucose levels were measured for 20 days. At day 20 after UTMD, the pancreas in each group was harvested and investigated with immunohistochemistry of insulin (beta cells) and CD31 (capillaries). Insulin positive beta cell mass in endogenous pancreas was measured as previously described [14]. The vessel density in the islets was measured as described above.

Statistical analysis: Data were expressed as mean±standard error of the mean (SEM). Statistical significance of the differences among the three groups was determined by ANOVA followed by Student's t-test with Bonferroni correction. Differences of ratio among 3 groups were analyzed by Tukey Honestly Significant Difference (HSD).

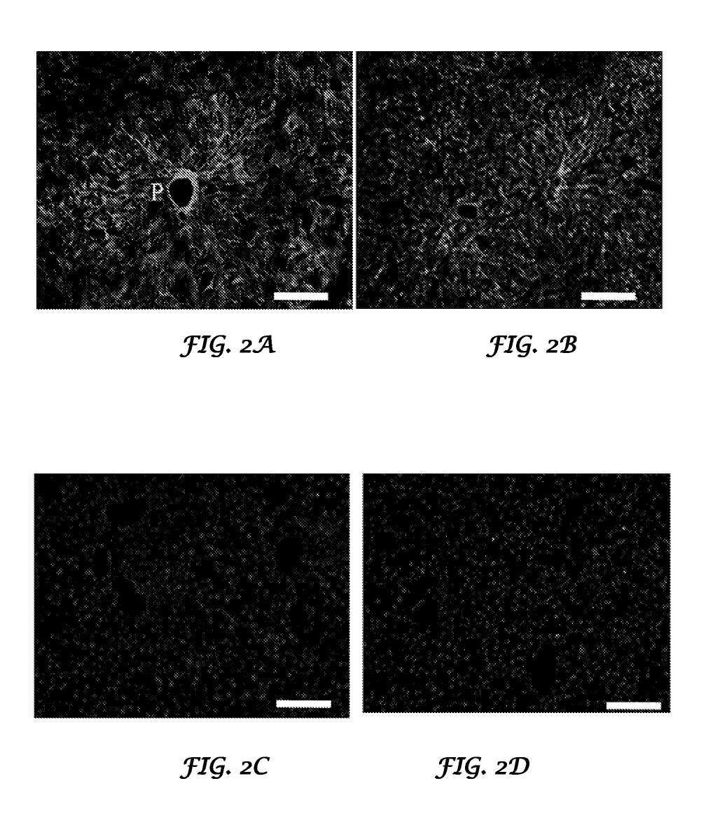

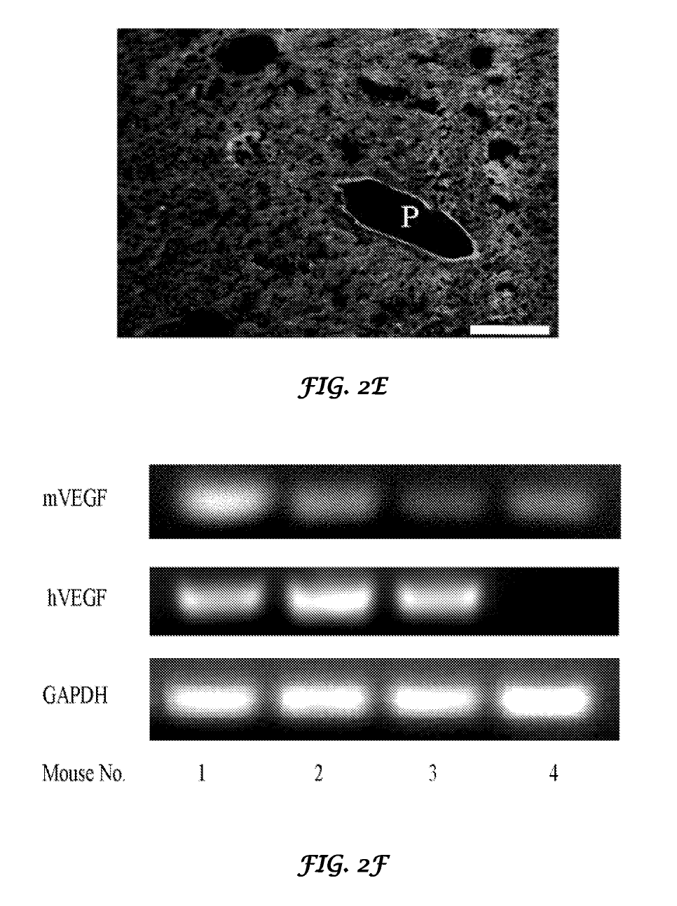

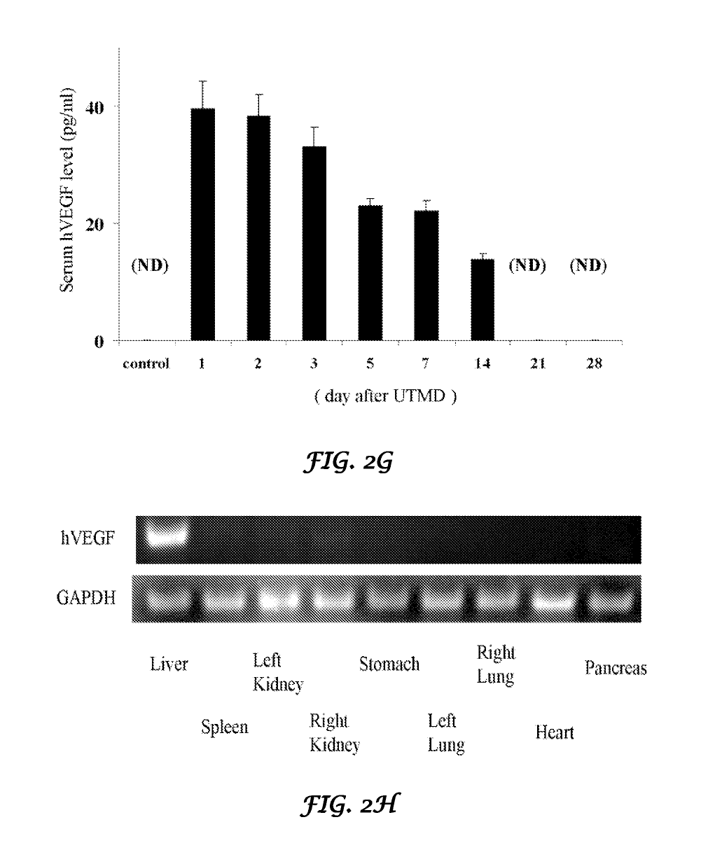

Effect of UTMD on gene delivery to liver via portal vein: UTMD with pCI-hVEGF and pCS2-GFP plasmids was applied to the liver of mice with intraportal injection of the microbubbles. At day 3, hVEGF (SEQ. ID NO: 10) or GFP expression was detected in the liver (FIG. 2). According to immunohistochemical analysis, hVEGF (SEQ. ID NO: 10) strongly expressed near portal veins, but the expression was distributed in a patchy fashion throughout the liver (FIGS. 2A-2C), whereas non-treated liver did not express hVEGF (SEQ. ID NO: 10) (FIG. 2D). GFP expression was detected in GFP-induced mouse liver (FIG. 2E). RT-PCR analysis showed that mouse VEGF (mVEGF) was expressed in normal mouse liver; however, hVEGF (SEQ. ID NO: 10) was never expressed. On the other hand, in the hVEGF-induced mice, hVEGF expression (SEQ. ID NO: 10) was clearly detected (FIG. 2F).

Serum hVEGF (SEQ. ID NO: 10) level after UTMD: Initial studies by the inventors using pCI-hVEGF in vitro proved that hVEGF protein (SEQ. ID NO: 10) was secreted from the transfected cells into culture media (data not shown). The inventors evaluated hVEGF (SEQ. ID NO: 10) level in the blood of the mice treated with UTMD. hVEGF (SEQ. ID NO: 10) was detected for 14 days after UTMD, although the levels were low (FIG. 2G). This might be because of the effect of dilution and decomposition by blood.

Organ specificity of gene delivery: Various organs were harvested from the mice 3 days after UTMD via portal vein and the hVEGF expression (SEQ. ID NO: 10) in each organ was examined by RT-PCR. hVEGF (SEQ. ID NO: 10) was strongly expressed in the liver, whereas other organs showed no expression (FIG. 2H). The right kidney slightly expressed hVEGF, probably because it was exposed to ultrasound due to its anatomical proximity to liver.

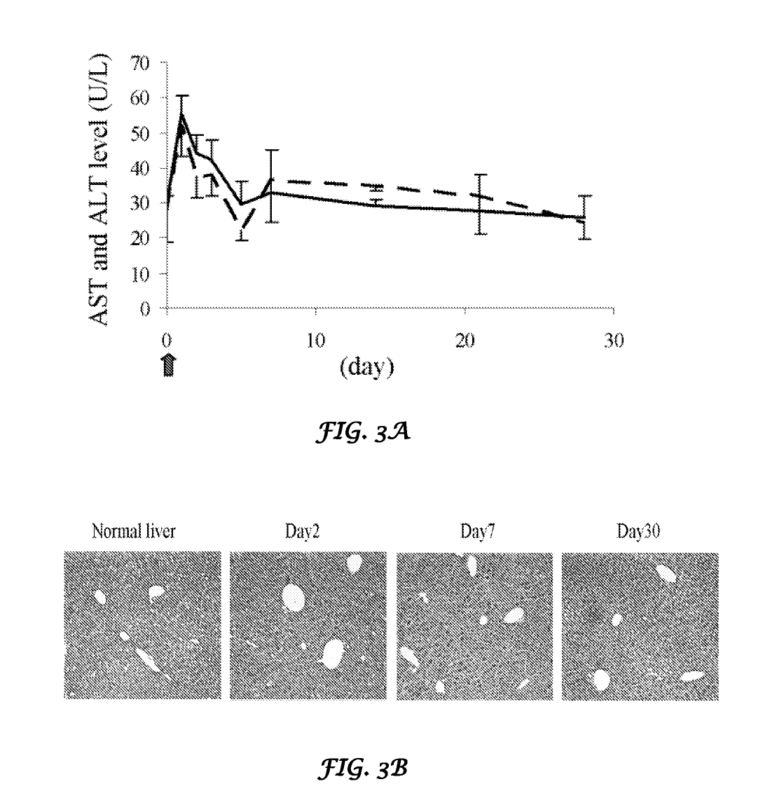

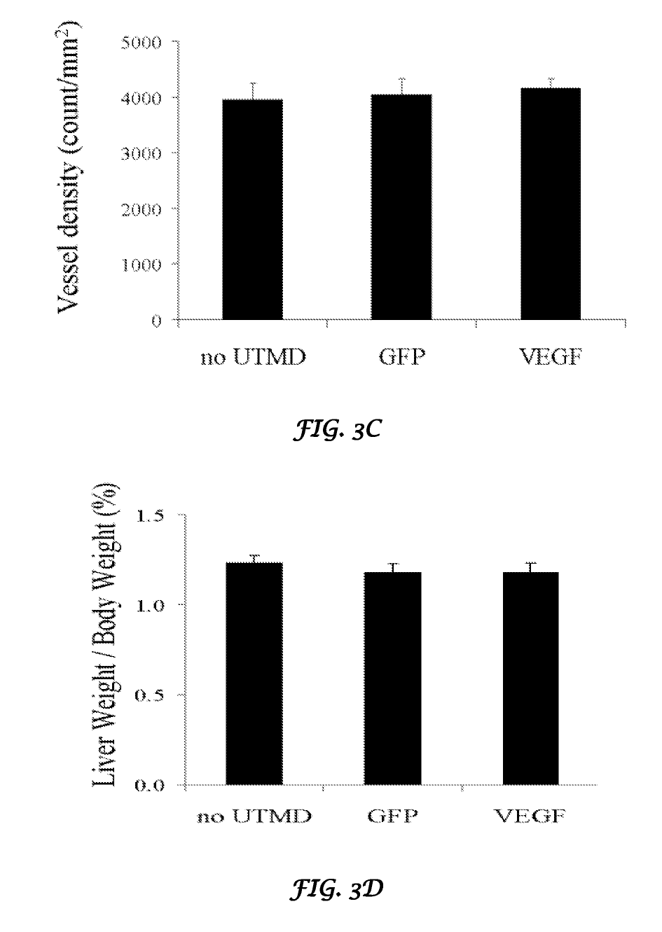

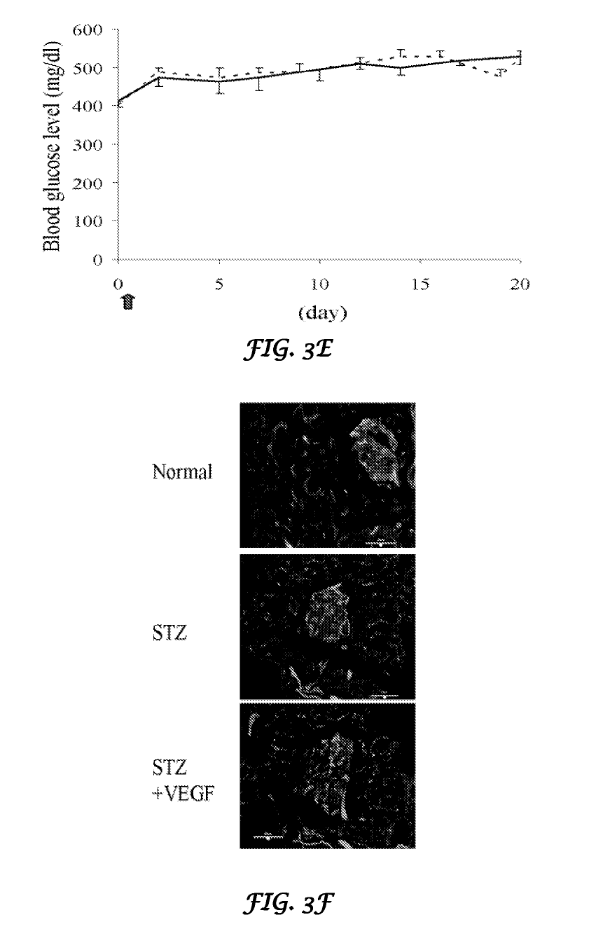

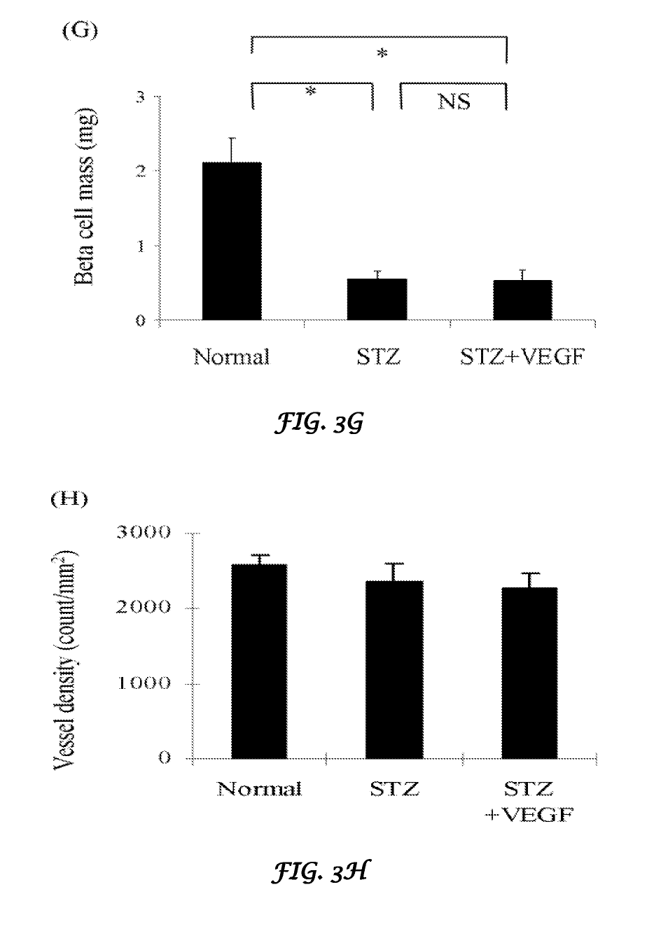

Effect of UTMD and hVEGF on Liver: The toxicity in the mouse livers caused by UTMD was examined. Serum ALT and AST levels were measured after UTMD. Both ALT and AST levels were slightly elevated at the first day after UTMD and then rapidly declined to normal levels (FIG. 3A). These data indicated that toxicity was trifling and transient. To further determine the extent of hepatic injury following UTMD, the livers were histologically examined at day 2, 7, 30 days after UTMD. Although very slight disruption of hepatocyte architecture was observed at day 2, the majority of the hepatic parenchyma did not show significant histological abnormality (FIG. 3B).

To examine whether local expression of hVEGF (SEQ. ID NO: 10) could increase the vessels in liver or cause uncontrollable angiogenesis and liver growth, the vessel density in liver and the weight of liver (left lobe) were measured. There were no remarkable differences in 3 groups (no UTMD, GFP and VEGF, FIGS. 3C and 3D).

Effect of circulating hVEGF on endogenous pancreas: As shown in FIG. 2, there was not hVEGF (SEQ. ID NO: 10) expression in pancreas after UTMD, but hVEGF (SEQ. ID NO: 10) was detected in serum. The inventors studied whether this low level hVEGF (SEQ. ID NO: 10) could affect endogenous pancreatic beta cells and islet vasculature. hVEGF gene (SEQ. ID NO: 9) delivery to liver did not influence blood glucose level (FIG. 3E), endogenous beta cell mass and vessel density in islets (FIGS. 3F-3H) for STZ-induced diabetic mice.

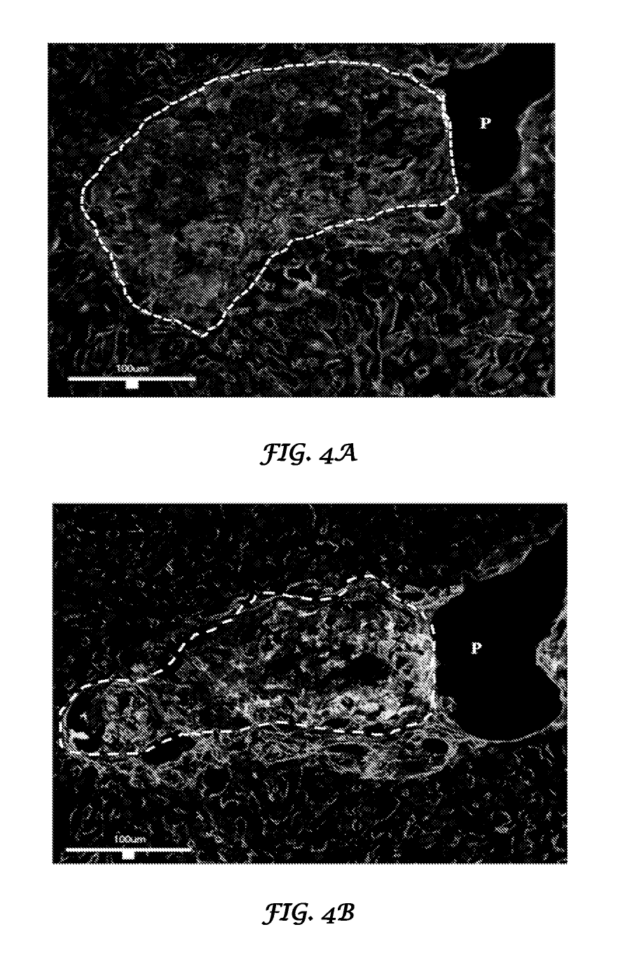

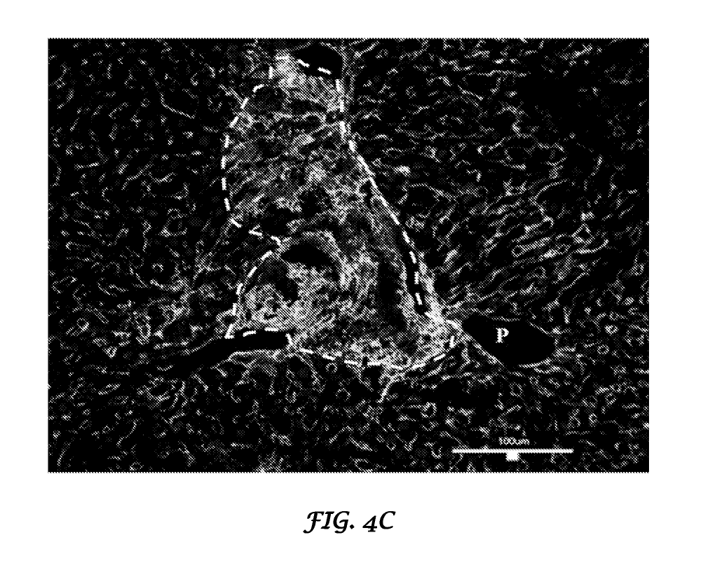

hVEGF expression in and around graft islets: In the present procedure, islet transplantation and gene delivery by UTMD were performed consecutively through the same route (portal vein). Therefore, it was expected that the induced gene could be efficiently delivered to graft islets and the surrounded tissues. With immunohistochemistry, hVEGF (SEQ. ID NO: 10) was detected in the periphery and the surrounding tissues of the islets, confirming successful transfer and expression of the exogenous angiogenic gene, but not in the center of islets (FIG. 4). Moreover, very few insulin positive beta cells doubly expressed hVEGF (SEQ. ID NO: 10) (FIG. 4A). Some of glucagon positive cells co-expressed hVEGF (SEQ. ID NO: 10) (FIG. 4B), whereas there were many double positive cells of hVEGF and Vimentin (FIG. 4C). These data indicate that hVEGF gene (SEQ. ID NO: 9) was mostly expressed by mesenchymal cells.

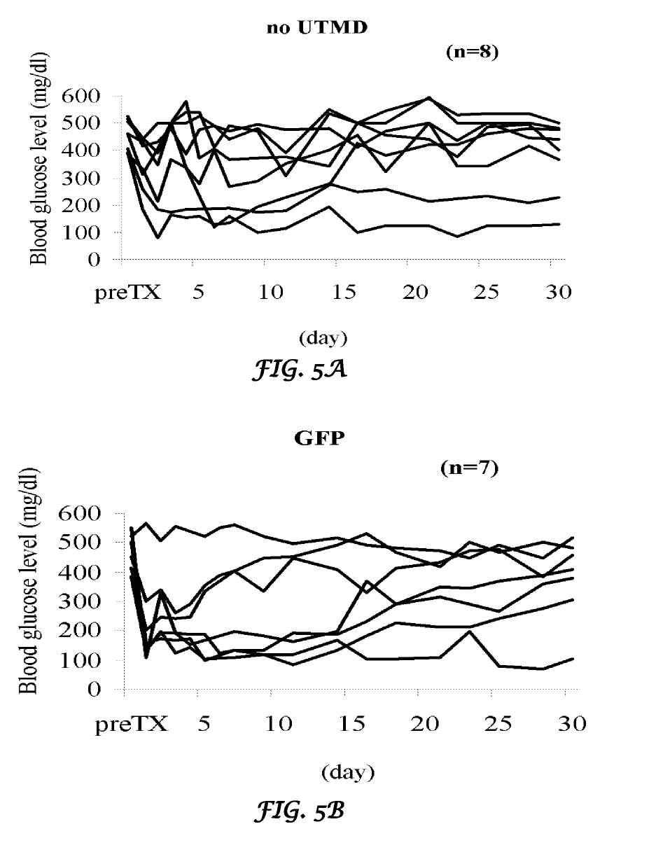

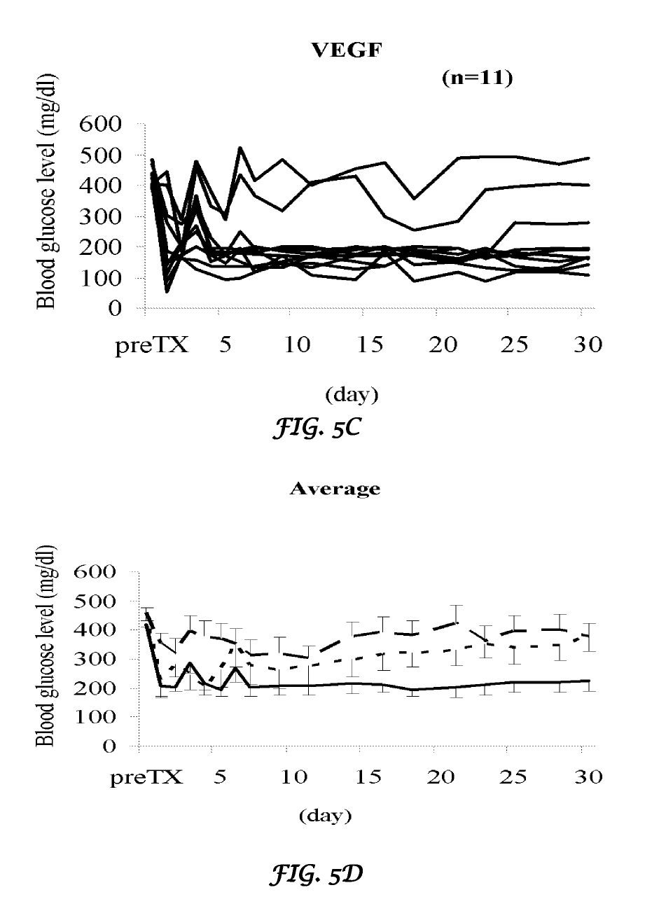

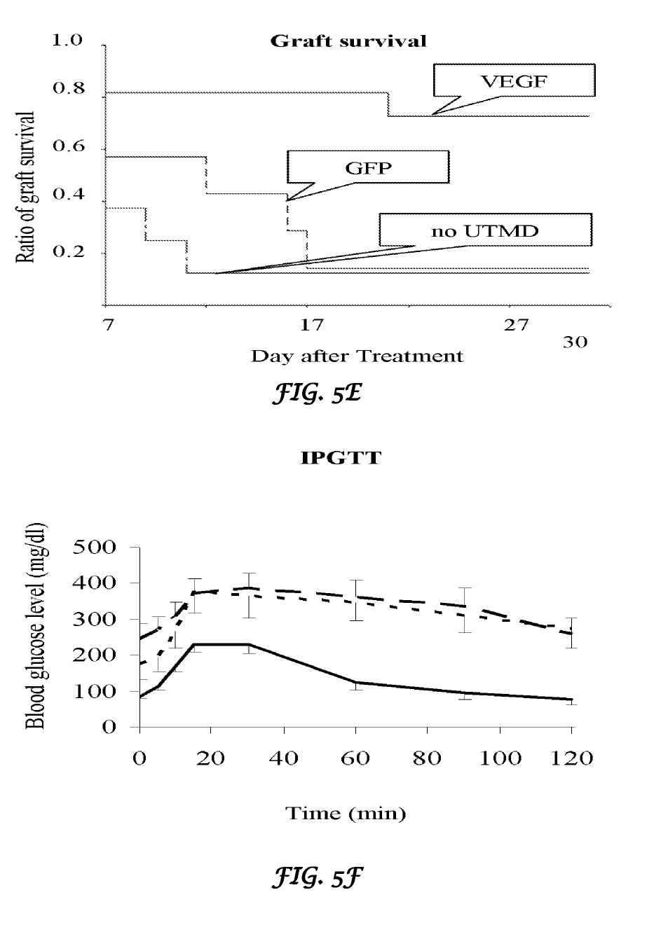

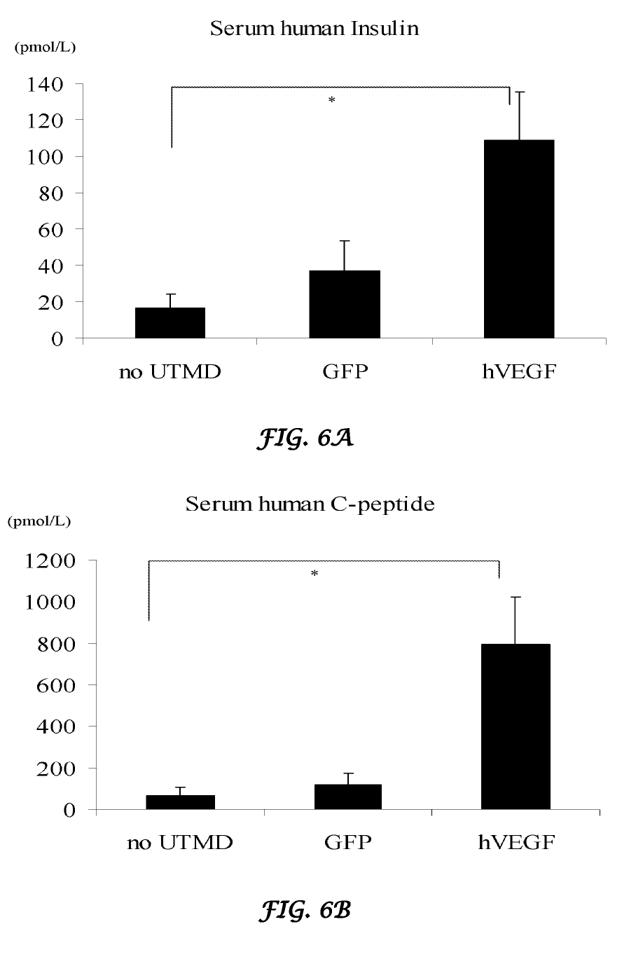

Effect of induced hVEGF on islet function after transplantation: The effect of hVEGF (SEQ. ID NO: 10) on the islet function after transplantation was determined in terms of daily blood glucose, human insulin and C-peptide levels at day 32. At post transplantation day 7, 9 out of 11 (82%) mice in hVEGF group became euglycemic whereas 3 out of 8 (38%) in no UTMD and 4 out of 7 (57%) in GFP group became euglycemic. However, most of the mice in no UTMD and GFP groups showed gradual increase in the blood glucose level and recurrence of diabetes over 30 days (FIG. 5). Overall, 8 out of 11 (73%) mice in hVEGF group became persistently euglycemic whereas 1 out of 8 (13%) in the control and 1 out of 7 (14%) in GFP group were euglycemic at day 30 (FIG. 5A-5D). The euglycemia rate of VEGF group at day 30 was significantly higher than no UTMD group (Table 1, p<0.05). The Kaplan-Meier estimate showed the graft survival rate of VEGF group was significantly higher than the other 2 groups (FIG. 5E, p<0.05 in no UTMD vs. UTMD and GFP vs. UTMD). The average serum human insulin and C-peptide levels in the hVEGF group were significantly higher than no UTMD group (FIG. 6: insulin=no UTMD: 16.6±7.6 pmol/l, GFP: 37.0±16.5 pmol/l, VEGF: 108.9±26.4 pmol/l, p=0.013 in no UTMD vs. VEGF; C-peptide=no UTMD: 68.4±37.5 pmol/l, GFP: 115.3±58.4 pmol/l, VEGF: 791.5±230.5 pmol/l, p=0.022 in no UTMD vs. VEGF).

Glucose tolerance test: To further define the function of the transplanted islets, IPGTT was performed at day 31 post-transplantation. The 2 hour blood glucose levels in both the no UTMD and GFP groups were more than 200 mg/dl, whereas the VEGF group showed a normal pattern (FIG. 5F).

TABLE 1
The rate of euglycemia 30 days after treatment in 3 groups.
Differences among 3 groups were analyzed by Tukey HSD.

| | no UTMD | GFP | VEGF |
|---|---|---|---|
| Transplant | 8 | 7 | 11 |
| Euglycemia | 1 | 1 | 8 |
| % | 12.5^{a} | 14.3 | 72.7^{a} |

^{a}p < 0.05 in noUTMD vs. VEGF.

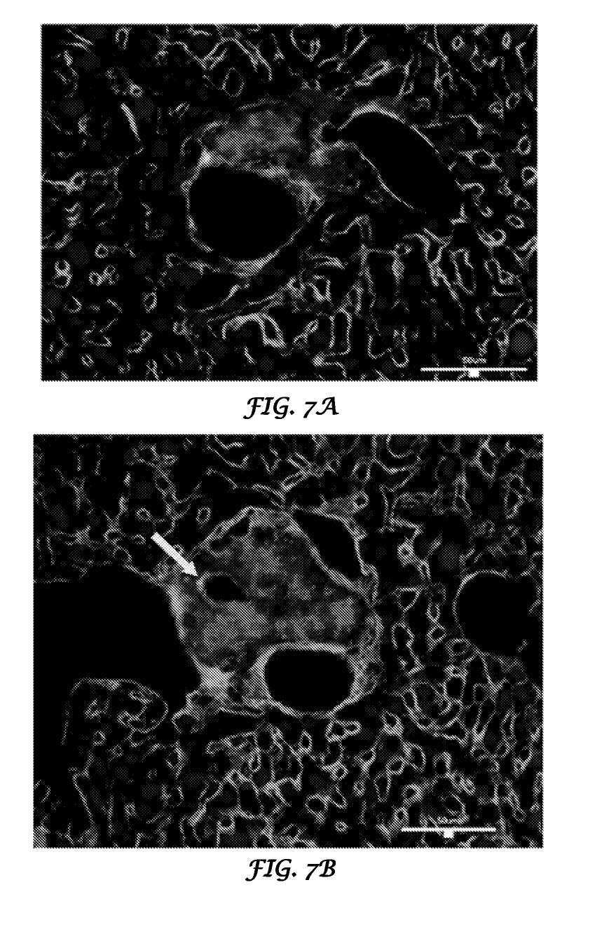

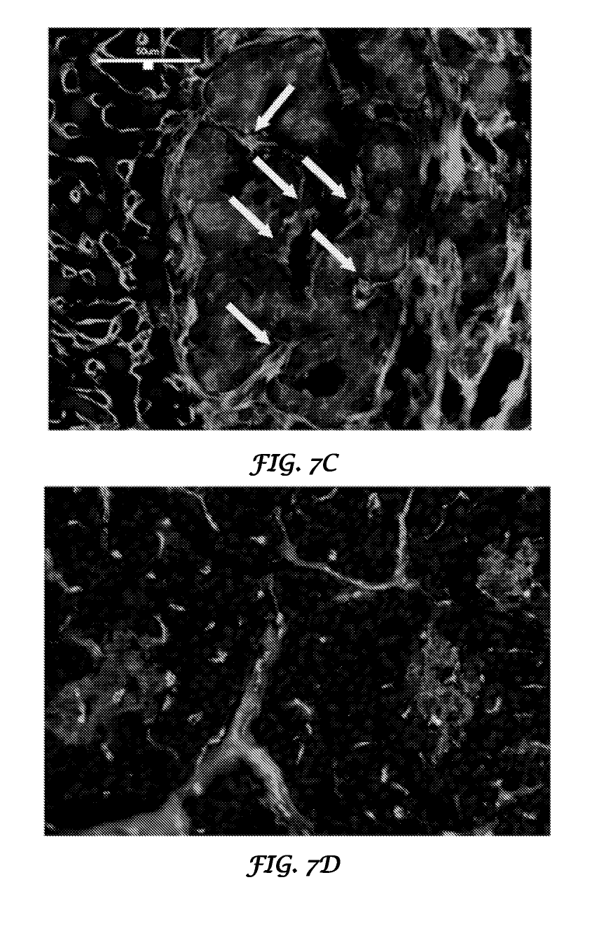

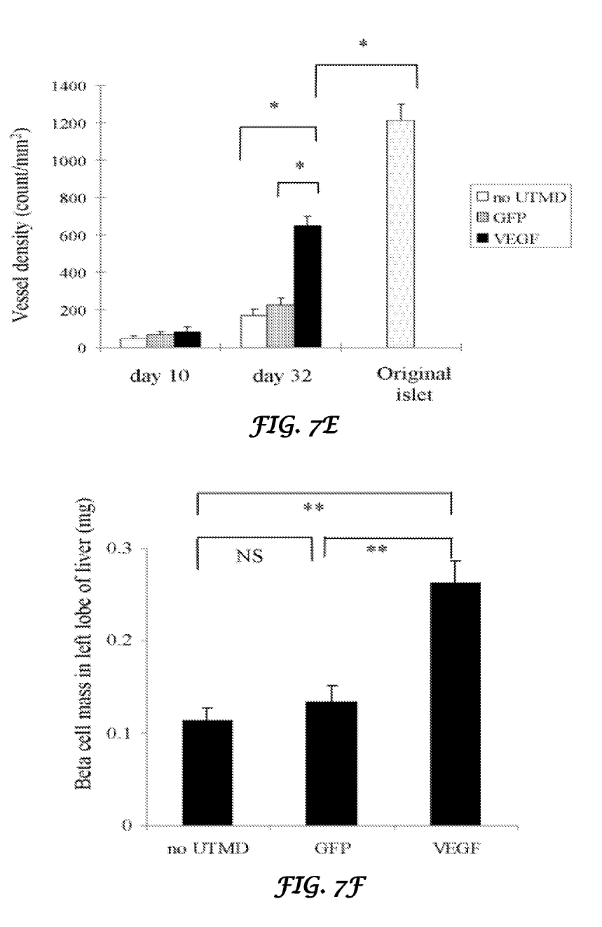

Intra-islet vascularization and beta cell mass: At 32 days post-treatment, capillary density in the transplanted islets was evaluated with immunohistochemical analysis (FIGS. 7A-7E). The islets were visualized with anti-insulin antibody, and the intra-islet vessels were stained using both anti human CD31 and anti mouse PECAM-1 antibodies, since Brissova et al. reported intra-islet endothelial cells contribute to revascularization of transplanted islets as well as host endothelial cells [15]. In the hVEGF treated mice, capillary density was significantly higher than in both of the control groups (FIG. 7E: no UTMD; 169±36, GFP; 227±39, VEGF; 649±51 count/mm², respectively. p<0.0001 in no UTMD vs. VEGF and GFP vs. VEGF). The vessel density in hVEGF group clearly increased compared to no UTMD group, however, it was still significantly lower than the natural human islets in pancreas (Original islets; 1215±86 count/mm² p<0.00001 in VEGF vs. Original islets). Beta cell mass in the left lobe of liver at day 32 after treatment was significantly greater in VEGF group than other groups (FIG. 7F: no UTMD; 0.11±0.01, GFP; 0.13±0.02, VEGF; 0.26±0.02, respectively. p<0.005 in noUTMD vs. VEGF and GFP vs. VEGF).

Revascularization to the transplanted islets is essential to improve their survival [16-19]. It was reported that pancreatic islet production of VEGF is critical for islet vascularization and function [20]. To promote the revascularization of the transplanted islets, ex vivo transduction of islets with an adenoviral vector encoding hVEGF has been examined by the present inventors with evidence of revascularization and improved islet survival [3, 21-23]. However, viral gene therapy is associated with severe adverse events [7, 8]. Hydrodynamics-based delivery of naked pDNA showed a therapeutic effect [22], nevertheless its procedure is clinically unsuitable. It was shown that simple injection of pDNA alone without hydrodynamic pressure is ineffective [24]. At first, the inventors tested the infusion of naked hVEGF plasmids via portal vein without microbubbles and ultrasound because this is the simplest method. However, the gene expression was not detected in liver (data not shown). Then, the inventors examined the UTMD method of the present invention, which has been shown to be highly effective without significant adverse bioeffect [9-11, 25-28].

In the present study, the inventors employed a clinical relevant intraportal islet transplantation model. Furthermore, the inventors injected the microbubbles with pDNA from the portal vein continuously after islets infusion. Intraportal injection of microbubbles has several advantages. First, the inventors could directly deliver a highly concentrated pDNA to liver compared to intravenous injection that was shown more effective than intrahepatic delivery [29]. Second, it could result in efficient gene delivery to both the graft islet and the surrounding tissues. Obviously this could increase total gene expression compared to gene delivery only to islets, and it would be another advantage of UTMD over ex vivo gene delivery. For the purpose, the inventors used CMV promoter instead of rat insulin promoter, which has was shown to be effective in specifically directing genes to islets by UTMD.

The findings provide evidence that hVEGF gene (SEQ. ID NO: 9) can be targeted noninvasively to the liver and the transplanted islets, and can modify the intra-islet microvasculature. Notably, the present invention is the first evidence that noninvasive delivery of a transgene to the host liver has therapeutic potential and it can produce biological changes in the vascularization of graft islets. Even though UMTD is noninvasive procedure, this method may damage the liver. To assess the damage of liver, we examined transaminases levels and histology of liver after UMTD. Those examinations revealed that the adverse effect of UMTD to liver was minimal.

The increased microvasculature in graft islets likely contributed to the improvement of efficacy of islet transplantation. However, considering the time course of graft islet loss and VEGF expression and the extent of revascularization, there might be the possibility of another mechanism of the effect of VEGF on the improved engraftment. In the present study, the capillary density increased in the transplanted islets 32 days after hVEGF plasmid gene transfer. Nevertheless, induced hVEGF protein (SEQ. ID NO: 10) was detectable in the treated mice only for 14 days in blood and for 10 days in liver. The inventors have previously reported that hVEGF delivery to heart in rat by UTMD increased capillary density but a regression of capillary density to the baseline level was observed by day 30, probably because of the transient nature of the plasmid expression [9]. Since upregulated VEGF expression could lead to abnormal blood vessels and hemangiomas in islets [30], the transient hVEGF (SEQ. ID NO: 10) expression of the method of the present invention offers an additional advantage.

Ultrasonic microbubble destruction causes cavitation, thermal effects, microstreaming, free radical production and microcapillary ruptures [31-34], which might activate endothelial cells or other cells. Indeed, slight inflammation and disruption of hepatocyte architecture were observed in both GFP and hVEGF group (data not shown), although data from the present inventors shows very little liver dysfunction after UTMD. Islet transplantation itself causes severe inflammation; therefore, the potential adverse bioeffects of microbubble destruction appear to be offset by the therapeutic effects of gene therapy in the present study. To prevent the inflammation associated with islet transplantation, a combination use of hVEGF and anti-inflammation genes or drugs might be useful [35].

hVEGF gene (SEQ. ID NO: 9) delivery by UTMD to the transplanted islets and the host liver according to the method of the present invention leads to increasing vessel density in the graft islets and improving graft function. Gene delivery by UTMD is safe and effective to improve islet transplantation.

It is contemplated that any embodiment discussed in this specification can be implemented with respect to any method, kit, reagent, or composition of the invention, and vice versa. Furthermore, compositions of the invention can be used to achieve methods of the invention.

It may be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it may be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

U.S. Patent Application No. 20080114287: Ultrasound Microbubble Mediated Genes Delivery System.
U.S. Pat. No. 7,374,390: GLP-1 Gene Delivery for the Treatment of Type 2 Diabetes.
U.S. Patent Application No. 20090209630: Gene Delivery Formulations and Methods for Treatment of Ischemic Conditions.
1. Ryan E A, Paty B W, Senior P A et al (2005) Five-year follow-up after clinical islet transplantation. Diabetes 54:2060-2069
2. Berney T, Ricordi C (2000) Islet cell transplantation: the future? Langenbecks Arch Surg 385:373-378
3. Narang A S, Sabek O, Gaber A O, Mahato R I (2006) Co-expression of VEGF and IL-lreceptor antagonist improves human islet survival and function. Pharmaceutical Res 23:1970-1982
4. Fernandes J R, Duvivier-Kali V F, Keegan M et al (2004) Transplantation of islets transduced with CTLA4-Ig and TGFβ using adenovirus and lentivirus vectors. Transplant Immunology 13:191-200
5. Rehman K K, Bertera S, Trucco M, Gambotto A, Robbins PD (2007) Immunomodulation by adenoviral-mediated SCD40-Ig gene therapy for mouse allogeneic islet transplantation. Transplantation 84:301-307
6. Panakanti R, Mahato R I (2009) Bipartite adenoviral vector encoding hHGF and hIL-1Ra for improved human islet transplantation. Pharm Res 26:587-596
7. Hacein-Bey-Abina S, von Kalle C, Schmidt M et al (2003) A serious adverse event after successful gene therapy for X-linked severe combined immunodeficiency. N Engl J Med 348:255-256
8. Manno C S, Pierce G F, Arruda V R et al (2006) Successful transduction of liver in hemophilia by AAV-Factor IX and limitations imposed by the host immune response. Nat Med 12:342-347
9. Korpanty G, Chen S, Shohet R V et al (2005) Targeting of VEGF-mediated angiogenesis to rat myocardium using ultrasonic destruction of microbubbles. Gene Ther 12:1305-1312
10. Chen S, Ding J H, Bekeredjian R (2006) Efficient gene delivery to pancreatic islets with ultrasonic microbubble destruction technology. Proc Natl Acad Sci USA 103:8469-8474
11. Chen S, Ding J, Yu C (2007) Reversal of streptozotocin-induced diabetes in rats by gene therapy with betacellulin and pancreatic duodenal homeobox-1. Gene Ther 14:1102-1110
12. Matsumoto S, Noguchi H, Shimoda M et al (2009) Seven consecutive successful clinical islet isolations with pancreatic ductal injection. Cell Transplant in press
13. Matsumoto S, Okitsu T, Iwanaga Y et al (2006) Successful islet transplantation from non-heart-beating donor pancreata using modified Ricordi islet isolation method. Transplantation 82:460-465
14. Montaña E, Bonner-Weir S, Weir G C (1993) Beta cell mass and growth after syngeneic islet cell transplantation in normal and streptozocin diabetic C57BL/6 mice. J Clin Invest 91:780-787
15. Brissova M, Fowler M, Wiebe P (2004) Intraislet endothelial cells contribute to revascularization of transplanted pancreatic islets. Diabetes 53:1318-1325
16. Narang A S, Mahato R I (2006) Biological and biomaterial approaches for improved islet transplantation. Pharmacol Rev 58:194-243
17. Jones G L, Juszczak M T, Hughes S J, Kooner P, Powis S H, Press M (2007) Time course and quantification of pancreatic islet revascularization following intraportal transplantation. Cell Transplant 16:505-516
18. Menger M D; Vajkoczy P, Beger C, Messmer K (1994) Orientation of microvascular blood flow in pancreatic islet isografts. J Clin Invest 93:2280-2285
19. Vajkoczy P, Olofsson A M, Lehr H A (1995) Histogenesis and ultrastructure of pancreatic islet graft microvasculature. Evidence for graft revascularization by endothelial cells of host origin. Am J Pathol 146:1397-1405
20. Brissova M, Shostak A, Shiota M (2006) Pancreatic islet production of vascular endothelial growth factor-A is essential for islet vascularization, revascularization, and function. Diabetes 55:2974-2985
21. Narang A S, Cheng K, Henry J (2004) Vascular endothelial growth factor gene delivery for revascularization in transplanted human islets. Pharm Res 21:15-25
22. Cheng K, Fraga D, Zhang C (2004) Adenovirus-based vascular endothelial growth factor gene delivery to human pancreatic islets. Gene Ther 11:1105-1116.
23. Zhang N, Richter A, Suriawinata J (2004) Elevated vascular endothelial growth factor production in islets improves islet graft vascularization. Diabetes 53:963-970
24. Miao C H, Thompson A R, Loeb K, Ye X (2001) Long-term and therapeutic-level hepatic gene expression of human factor IX after naked plasmid transfer in vivo. Mol Ther 3:947-957
25. Bekeredjian R, Chen S, Pan W, Grayburn P A, Shohet R V (2004) Effects of ultrasound-targeted microbubble destruction on cardiac gene expression. Ultrasound Med Biol 30:539-543
26. Bekeredjian R, Chen S, Frenkel P A, Grayburn P A, Shohet R V (2003) Ultrasound-targeted microbubble destruction can repeatedly direct highly specific plasmid expression to the heart. Circulation 108:1022-1026
27. Chen S, Shohet R V, Bekeredjian R, Frenkel P, Grayburn P A (2003) Optimization of ultrasound parameters for cardiac gene delivery of adenoviral or plasmid deoxyribonucleic acid by ultrasound-targeted microbubble destruction. J Am Coll Cardiol 42:301-308
28. Chen S, Kroll M H, Shohet R V, Frenkel P, Mayer S A, Grayburn P A (2002) Bioeffects of myocardial contrast microbubble destruction by echocardiography. Echocardiography 19:495-500
29. Shen Z P, Brayman A A, Chen L, Miao C H (2008) Ultrasound with microbubbles enhances gene expression of plasmid DNA in the liver via intraportal delivery. Gene Ther 15:1147-1155
30. Ozawa C R, Banfi A, Glazer N L (2004) Microenvironmental VEGF concentration, not total dose, determines a threshold between normal and aberrant angiogenesis. J Clin Invest 113:516-527
31. Miller D L, Gies R A (1998) The interaction of ultrasonic heating and cavitation in vascular bioeffects on mouse intestine. Ultrasound Med Biol 24:123-128
32. Wu J, Ross J P, Chiu J F (2002) Reparable sonoporation generated by microstreaming. J Acoust Soc Am 111:1460-4
33. Kondo T, Misik V, Riesz P (1998) Effect of gas-containing microspheres and echo contrast agents on free radical formation by ultrasound. Free Radic Biol Med 25:605-612
34. Skyba D M, Price R J, Linka A Z, Skalak T C, Kaul S (1998) Direct in vivo visualization of intravascular destruction of microbubbles by ultrasound and its local effects on tissue. Circulation 98:290-293
35. Panakanti R, Mahato R I (2009) Bipartite vector encoding hVEGF and hIL-1Ra for ex vivo transduction into human islets. Mol Pharm 6:274-284

What is claimed is:

1. A composition for ultrasound-targeted microbubble destruction (UTMD) in one or more liver cells, a liver or an islet cell transplanted into the liver comprising:
one or more pre-assembled liposome plasmid DNA (pDNA) microbubble complexes, wherein the microbubble comprises a lipid shell enclosing a gas and a pDNA comprising a constitutive promoter sequence or an inducible promoter sequence operably linked to a human vascular endothelial growth factor (hVEGF), wherein an ultrasound disruption of the one or more microbubbles in the one or more liver cells, the liver or the islet cells transplanted into the liver delivers the pDNA into the one or more liver cells, the liver or the islet cells transplanted into the liver at a location of the ultrasound disruption to express hVEGF, wherein the composition improves the efficacy of the one or more transplanted islet cells.

2. The composition of claim 1, wherein the lipid shell comprises one or more additional bioactive agents selected from the group consisting of naked DNA, siRNA, plasmids, proteins, viral vectors, and drugs.

3. The composition of claim 1, wherein the gas is a perfluorocarbon gas.

4. The composition of claim 1, wherein the inducible promoter comprises a tissue-specific regulatory element.

5. The composition of claim 1, wherein the efficacy of the islet transplantation is measured by improved revascularization, improved islet cell function, increased vessel density or combinations thereof.

6. The composition of claim 1, wherein the hVEGF is a recombinant hVEGF.

7. The composition of claim 1, wherein one or more agents may be co-administered with the composition, wherein the agents are selected from the group consisting of an anti-apoptotic agent, an anti-inflammatory agent, a JNK inhibitor, a GLP-1, a tacrolimus, a sirolimus, an anakinra, a Dervin polyamide or combinations thereof.

8. A composition for regenerating transplanted islet cells in a liver or a transplanted liver using ultrasound-targeted microbubble destruction (UTMD) comprising:
microbubbles comprising a naked plasmid DNA encoding a human vascular endothelial growth factor (hVEGF), wherein the microbubbles comprise lipids that release the hVEGF by ultrasound disruption in the liver or the transplanted liver.

9. The composition of claim 8, wherein the hVEGF is a recombinant hVEGF.

10. The composition of claim 8, wherein the hVEGF is under the control of a cytomegalovirus (CMV) promoter.

11. A method for promoting revascularization, improving function, increasing vessel density and efficacy of one or more transplanted cells or grafted cells in vivo and in situ in subject comprising the step of:
delivering an effective amount of a microbubble composition comprising a naked plasmid DNA encoding a human vascular endothelial growth factor (hVEGF), wherein the microbubbles comprise lipids that release the hVEGF by an ultrasound disruption in the one or more transplanted or grafted cells, wherein the released hVEGF promotes revascularization, improves function, vessel density and efficacy of the one or more transplanted or grafted cells.

12. The method of claim 11, wherein the one or more transplanted or grafted cells comprise islet cells.

13. The method of claim 11, wherein the subject is a healthy subject, a diabetic subject or a subject in need of one or more transplanted or grafted cells.

14. A method of improving vascularization, increasing vessel density and efficacy of one or more transplanted islet cells in a liver of a patient comprising the steps of:
injecting the patient with a naked plasmid DNA microbubble complex comprising a plasmid expressing a human vascular endothelial growth factor (hVEGF) gene under the control of cytomegalovirus (CMV) promoter, wherein the injection is done in the liver of the patient;
delivering the pDNA to the one or more transplanted islet cells in the liver; and
maintaining the one or more transplanted islet cells under conditions effective to express the hVEGF gene, wherein the expression of the hVEGF causes improved vascularization, increased vessel density and efficacy of the one or more transplanted islet cells.

15. The method of claim 14, further comprising the step of optional co-administration of one or more agents, wherein the agents are selected from the group consisting of an anti-apoptotic agent, an anti-inflammatory agent, a JNK inhibitor, a GLP-1, a tacrolimus, a sirolimus, an anakinra, a Dervin polyamide or combinations thereof.

16. The method of claim 14, wherein the microbubble comprises a pre-assembled liposome-naked plasmid DNA (pDNA) complex.

17. The method of claim 14, wherein the microbubble comprises a pre-assembled liposome-pDNA complex that comprises 1,2-dipalmitoyl-sn-glycero-3-phosphatidylcholine and 1,2-dipalmitoyl-sn-glycero-3-phosphatidylethanolamine glycerol mixed with a plasmid.

18. A method of treating diabetes or promoting euglycemia in a patient comprising the steps of:
identifying the patient in need of treatment against the diabetes or promotion of the euglycemia;
transplanting one or more islet cells by infusing the patient's liver with one or more islet cells, wherein the one or more transplanted islet cells produce insulin for the treatment of the diabetes or for the promotion of the euglycemia;
injecting an effective amount of a microbubble composition comprising a naked plasmid DNA (pDNA) encoding a human vascular endothelial growth factor (hVEGF), wherein the microbubbles comprise lipids that release the hVEGF by an ultrasound disruption in the one or more transplanted islet cells, wherein the released hVEGF promotes revascularization, improves function, vessel density, and efficacy of the one or more transplanted islet cells; and
treating the diabetes or promoting the euglycemia by the production of insulin by the one or more transplanted islet cells.

19. The method of claim 18, wherein the hVEGF is a recombinant hVEGF.

20. The method of claim 18, wherein the hVEGF is under the control of a cytomegalovirus (CMV) promoter.

21. The method of claim 18, further comprising optional co-administration of one or more agents, wherein the agents are selected from the group consisting of an anti-apoptotic agent, an anti-inflammatory agent, a JNK inhibitor, a GLP-1, a tacrolimus, a sirolimus, an anakinra, a Dervin polyamide or combinations thereof.

22. A composition for ultrasound-targeted microbubble destruction (UTMD) in a body organ comprising:
a pre-assembled liposome-bioactive agent complex in contact with a microbubble, wherein the bioactive agents are selected from the group consisting of a naked plasmid DNA (pDNA), a siRNA, one or more plasmids, proteins, viral vectors and drugs, wherein the pre-assembled liposome-bioactive agent complex may express a gene under the control of one or more promoters, wherein disruption of the microbubble with ultrasound in the body organ at a target site delivers the bioactive agent at a location of the ultrasound disruption.

23. The composition of claim 22, wherein the one or more cells comprise transplanted islet cells.

24. The composition of claim 22, wherein the body organs comprise liver, pancreas, kidney, lungs or heart.

25. The composition of claim 22, wherein the body organ is the liver.

26. The composition of claim 22, wherein the bioactive agent is a pDNA.

27. The composition of claim 22, wherein the pre-assembled liposome-bioactive agent complex expresses a recombinant human vascular endothelial growth factor (hVEGF) gene under the control of a cytomegalovirus (CMV) promoter.

28. The composition of claim 22, wherein the pre-assembled liposome-nucleic acid complex comprises cationic lipids, anionic lipids or mixtures and combinations thereof.

29. The composition of claim 22, wherein the microbubbles are disposed in a pharmaceutically acceptable vehicle.

30. The composition of claim 22, wherein the pre-assembled liposome-bioactive agent complex comprise 1,2-dipalmitoyl-sn-glycero-3-phosphatidylcholine and 1,2-dipalmitoyl-sn-glycero-3-phosphatidylethanolamine glycerol mixed with a plasmid.

31. The composition of claim 22, wherein the composition promotes revascularization, improves function, increases vessel density and efficacy of the one or more transplanted islet cells.

32. The composition of claim 22, further comprising an optional coating.


SEQUENCE LISTING (12 sequences)

SEQ ID NO: 1 - DNA, 33 bp, Artificial Sequence
  Synthetic oligonucleotide.
ttcctcgagaatgaactttctgctgctgtcttg

SEQ ID NO: 2 - DNA, 28 bp, Artificial Sequence
  Synthetic oligonucleotide.
aaacccgggtcaccgcctcggcttgtca

SEQ ID NO: 3 - DNA, 24 bp, Artificial Sequence
  Synthetic oligonucleotide.
cccttcattgacctcaactacatg

SEQ ID NO: 4 - DNA, 22 bp, Artificial Sequence
  Synthetic oligonucleotide.
ttccattgatgacaagcttccc

SEQ ID NO: 5 - DNA, 20 bp, Artificial Sequence
  Syntehtic oligonucleotide.
aaggaggagggcagaatcat

SEQ ID NO: 6 - DNA, 20 bp, Artificial Sequence
  Synthetic oligonucleotide.
atctgcatggtgatgttgga

SEQ ID NO: 7 - DNA, 22 bp, Artificial Sequence
  Synthetic oligonucleotide.
acgacagaaggagagcagaagt

SEQ ID NO: 8 - DNA, 22 bp, Artificial Sequence
  Synthetic oligonucleotide.
catggtgatgttgctctctgac

SEQ ID NO: 9 - DNA, 3392 bp, Homo sapiens
tcgcggaggcttggggcagccgggtagctcggaggtcgtggcgctgggggctagcaccagcgctctgtcgggaggcgcagcggttaggtggaccggtcagcggactcaccggccagggcgctcggtgctggaatttgatattcattgatccgggttttatccctcttcttttttcttaaacatttttttttaaaactgtattgtttctcgttttaatttatttttgcttgccattccccacttgaatcgggccgacggcttggggagattgctctacttccccaaatcactgtggattttggaaaccagcagaaagaggaaagaggtagcaagagctccagagagaagtcgaggaagagagagacggggtcagagagagcgcgcgggcgtgcgagcagcgaaagcgacaggggcaaagtgagtgacctgcttttgggggtgaccgccggagcgcggcgtgagccctcccccttgggatcccgcagctgaccagtcgcgctgacggacagacagacagacaccgcccccagccccagctaccacctcctccccggccggcggcggacagtggacgcggcggcgagccgcgggcaggggccggagcccgcgcccggaggcggggtggagggggtcggggctcgcggcgtcgcactgaaacttttcgtccaacttctgggctgttctcgcttcggaggagccgtggtccgcgcgggggaagccgagccgagcggagccgcgagaagtgctagctcgggccgggaggagccgcagccggaggagggggaggaggaagaagagaaggaagaggagagggggccgcagtggcgactcggcgctcggaagccgggctcatggacgggtgaggcggcggtgtgcgcagacagtgctccagccgcgcgcgctccccaggccctggcccgggcctcgggccggggaggaagagtagctcgccgaggcgccgaggagagcgggccgccccacagcccgagccggagagggagcgcgagccgcgccggccccggtcgggcctccgaaaccatgaactttctgctgtcttgggtgcattggagccttgccttgctgctctacctccaccatgccaagtggtcccaggctgcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagatgtgacaagccgaggcggtgagccgggcaggaggaaggagcctccctcagggtttcgggaaccagatctctcaccaggaaagactgatacagaacgatcgatacagaaaccacgctgccgccaccacaccatcaccatcgacagaacagtccttaatccagaaacctgaaatgaaggaagaggagactctgcgcagagcactttgggtccggagggcgagactccggcggaagcattcccgggcgggtgacccagcacggtccctcttggaattggattcgccattttatttttcttgctgctaaatcaccgagcccggaagattagagagttttatttctgggattcctgtagacacacccacccacatacatacatttatatatatatatattatatatatataaaaataaatatctctattttatatatataaaatatatatattctttttttaaattaacagtgctaatgttattggtgtcttcactggatgtatttgactgctgtggacttgagttgggaggggaatgttcccactcagatcctgacagggaagaggaggagatgagagactctggcatgatcttttttttgtcccacttggtggggccagggtcctctcccctgcccaggaatgtgcaaggccagggcatgggggcaaatatgacccagttttgggaacaccgacaaacccagccctggcgctgagcctctctaccccaggtcagacggacagaaagacagatcacaggtacagggatgaggacaccggctctgaccaggagtttggggagcttcaggacattgctgtgctttggggattccctccacatgctgcacgcgcatctcgcccccaggggcactgcctggaagattcaggagcctgggcggccttcgcttactctcacctgcttctgagttgcccaggagaccactggcagatgtcccggcgaagagaagagacacattgttggaagaagcagcccatgacagctccccttcctgggactcgccctcatcctcttcctgctccccttcctggggtgcagcctaaaaggacctatgtcctcacaccattgaaaccactagttctgtccccccaggagacctggttgtgtgtgtgtgagtggttgaccttcctccatcccctggtccttcccttcccttcccgaggcacagagagacagggcaggatccacgtgcccattgtggaggcagagaaaagagaaagtgttttatatacggtacttatttaatatccctttttaattagaaattaaaacagttaatttaattaaagagtagggttttttttcagtattcttggttaatatttaatttcaactatttatgagatgtatcttttgctctctcttgctctcttatttgtaccggtttttgtatataaaattcatgtttccaatctctctctccctgatcggtgacagtcactagcttatcttgaacagatatttaattttgctaacactcagctctgccctccccgatcccctggctccccagcacacattcctttgaaataaggtttcaatatacatctacatactatatatatatttggcaacttgtatttgtgtgtatatatatatatatatgtttatgtatatatgtgattctgataaaatagacattgctattctgttttttatatgtaaaaacaaaacaagaaaaaatagagaattctacatactaaatctctctccttttttaattttaatatttgttatcatttatttattggtgctactgtttatccgtaataattgtggggaaaagatattaacatcacgtctttgtctctagtgcagtttttcgagatattccgtagtacatatttatttttaaacaacgacaaagaaatacagatatatcttaaaaaaaaaaaagcattttgtattaaagaatttaattctgatctcaaaaaaaaaaaaaaaaaa

SEQ ID NO: 10 - protein, 371 aa, Homo sapiens
MTDRQTDTAPSPSYHLLPGRRRTVDAAASRGQGPEPAPGGGVEGVGARGVALKLFVQLLGCSRFGGAVVRAGEAEPSGAARSASSGREEPQPEEGEEEEEKEEERGPQWRLGARKPGSWTGEAAVCADSAPAARAPQALARASGRGGRVARRGAEESGPPHSPSRRGSASRAGPGRASETMNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 11 - DNA, 714 bp, Aequorea victoria
atgagtaaaggagaagaacttttcactggagtggtcccagttcttgttgaattagatggcgatgttaatgggcaaaaattctctgtcagtggagagggtgaaggtgatgcaacatacggaaaacttacccttaattttatttgcactactgggaagctacctgttccatggccaacacttgtcactactttctcttatggtgttcaatgcttctcaagatacccagatcatatgaaacagcatgactttttcaagagtgccatgcccgaaggttatgtacaggaaagaactatattttacaaagatgacgggaactacaagacacgtgctgaagtcaagtttgaaggtgatacccttgttaatagaatcgagttaaaaggtattgattttaaagaagatggaaacattcttggacacaaaatggaatacaactataactcacataatgtatacatcatgggagacaaaccaaagaatggcatcaaagttaacttcaaaattagacacaacattaaagatggaagcgttcaattagcagaccattatcaacaaaatactccaattggcgatggccctgtccttttaccagacaaccattacctgtccacacaatctgccctttccaaagatcccaacgaaaagagagatcacatgatccttcttgagtttgtaacagctgctaggattacacatggcatggatgaactatacaaa

SEQ ID NO: 12 - protein, 238 aa, Aequorea victoria
MSKGEELFTGVVPVLVELDGDVNGQKFSVSGEGEGDATYGKLTLNFICTTGKLPVPWPTLVTTFSYGVQCFSRYPDHMKQHDFFKSAMPEGYVQERTIFYKDDGNYKTRAEVKFEGDTLVNRIELKGIDFKEDGNILGHKMEYNYNSHNVYIMGDKPKNGIKVNFKIRHNIKDGSVQLADHYQQNTPIGDGPVLLPDNHYLSTQSALSKDPNEKRDHMILLEFVTAARITHGMDELYK